(19) United States
(12) Patent Application Publication
(10) Pub. No.: US 2024/0032959 A1
(43) Pub. Date: Feb. 1, 2024

(54) WIRE FEEDING DEVICE

(71) Applicant: ASAHI INTECC CO., LTD., Seto-shi (JP)

(72) Inventors: Toshio DOI, Seto-shi (JP); Yusaku FUJITA, Seto-shi (JP); Tomoki YAMAGIWA, Seto-shi (JP)

(73) Assignee: ASAHI INTECC CO., LTD., Seto-shi (JP)

(21) Appl. No.: 18/379,984

(22) Filed: Oct. 13, 2023

Related U.S. Application Data
(63) Continuation of application No. PCT/JP2021/016082, filed on Apr. 20, 2021.

(51) Int. Cl. A61B 17/3207

(52) U.S. Cl. CPC A61B 17/3207; A61B 2017/320733; A61B 2017/00862

(57) ABSTRACT

A wire feeding device includes a grip portion that grips and releases the wire and moves in the distal end direction and a proximal end direction; a push spring that biases the grip portion toward the distal end direction; and a slider and a hook that deform the push spring and increase a biasing force. When the deformation of the push spring by the slider and the hook is released, the wire gripped by the grip portion is fed to the distal end direction by the biasing force from the push spring, and the wire feeding device is configured to be operated in (i) a first mode in which the wire is continuously fed in the distal end direction according to a first predetermined operation and (ii) a second mode in which the wire is fed once in the distal end direction according to a second predetermined operation.

### CROSS REFERENCE TO RELATED APPLICATION

This application is a “bypass” continuation application of International Application No. PCT/JP2021/016082, filed Apr. 20, 2021. The entire content of the priority application is incorporated herein by reference.

### TECHNICAL FIELD

The present disclosure relates to a wire feeding device that feeds a wire.

### BACKGROUND

To remove an occluding object that blocks a blood vessel, such as a Chronic Total Occlusion (CTO), and improve a blood flow, there is tested whether a soft guide wire can penetrate the occluding object, for example, and if the soft guide wire does not penetrate the occluding object, the guide wire is replaced gradually with a harder antegrade guide wire.

This method requires the effort to replace the guide wires and the cost to use a plurality of guide wires.

Further, to control the guide wire to penetrate an occluding object, a technician picks and operates a guide wire by hand. Thus, the guide wire feeding distance depends on the sense of the technician.

Meanwhile, the technique described in Japanese Patent Application Publication No. 2016-202711, for example, is known as a technique capable of feeding a medical wire with a given moving amount and preferably transmitting the pushing force of the medical wire.

### SUMMARY

In the technique described in Japanese Patent Application Publication No. 2016-202711, the technician feeds a medical wire by pressing a spring. For this reason, the pushing force against the medical wire is relatively limited and may not be sufficient for the medical wire to penetrate an occluding object. Thus, the medical wire needs to be replaced to enable penetration through the occluding object. Therefore, it is not possible to solve the problems of requiring the effort to replace the medical wires and the cost to use a plurality of medical wires.

Moreover, in the technique of the Japanese Patent Application Publication No. 2016-202711, when the grip member holding a medical wire moves to the distal end side exceeding a given moving amount, the engagement between the engagement member and the grip member, which maintains the gripped state of the medical wire, is cancelled so as to prevent a moving amount of the medical wire from exceeding the given moving amount. However, the moving amount of the medical wire may vary depending on the timing when the engagement between the engagement member and the grip member is cancelled.

The present disclosure has been made on the basis of the above-described circumstances, and an object of the present disclosure is to adjust a wire moving amount to an appropriate amount and feed the wire with appropriate force.

To achieve such an object, a wire feeding device according to a first aspect is a wire feeding device feeding a wire in a distal end direction, the wire feeding device includes a grip portion that is able to grip the wire and cancel a grip of the wire and is movable in the distal end direction and a proximal end direction, an elastic body that is able to energize the grip portion toward the distal end direction, and an energizing portion that deforms the elastic body and increases energizing force toward the distal end direction. In the wire feeding device, when deformation of the elastic body by the energizing portion is released, the wire gripped by the grip portion is fed to the distal end direction by the energizing force by the elastic body, and the wire feeding device is operable in a first mode in which the wire is continuously fed to the distal end direction according to a first predetermined operation and a second mode in which the wire is fed once in the distal end direction according to a second predetermined operation.

In the wire feeding device described above, in the first mode, a series of operations may be repeatedly performed according to the first predetermined operation in order of (a), (b), and (c): (a) gripping of the wire by the grip portion and deformation of the elastic body by the energizing portion, (b) releasing of the deformation of the elastic body and feeding of the grip portion gripping the wire to the distal end side by the energizing force by the elastic body, and (c) releasing the grip of the wire by the grip portion and movement of the grip portion to the proximal end side, and, in the second mode, (a) described above may be performed and then (b) described above may be performed according to the second predetermined operation.

The wire feeding device described above may further include an operation portion that is able to switch between the first mode and the second mode.

The wire feeding device described above may further include a deformation maintaining portion that is able to maintain a deformation state of the elastic body that is in a predetermined state due to an increase in the energizing force by the energizing portion and a maintaining state change portion that is able to change to a state in which maintaining of the deformation state of the elastic body by the deformation maintaining portion is disabled, and may be configured such that the operation portion is able to set the maintaining state change portion to a state in which maintaining of the deformation state of the elastic body by the deformation maintaining portion is disabled, in a case where the deformation state of the elastic body that is in the predetermined state due to an increase in the energizing force by the energizing portion is maintained by the deformation maintaining portion, when the operation portion is operated to set the maintaining state change portion to a state in which maintaining of the deformation state of the elastic body by the deformation maintaining portion is disabled, the grip portion is moved to the distal end direction by the energizing force by the elastic body that is disabled to maintain the deformation state and the wire gripped by the grip portion is fed to the distal end direction.

In the wire feeding device described above, the wire feeding device may be operable in the first mode when the operation portion sets the maintaining state change portion to a state in which maintaining of the deformation state of the elastic body by the deformation maintaining portion is disabled and maintains the state.

The wire feeding device described above may further include an operation fixing portion that is able to fix the operation portion to the state in which the maintaining state change portion is operated to a state in which maintaining of the deformation state of the elastic body by the deformation maintaining portion is disabled.

The wire feeding device further include a grip operation portion that is able to operate a state of grip of the wire by the grip portion.

A wire feeding device according to a second aspect is a wire feeding device feeding a wire in a distal end direction, the wire feeding device includes a grip portion that is able to grip the wire and release a grip of the wire, and is movable in the distal end direction and a proximal end direction; an elastic body that is able to energize the grip portion toward the distal end direction; an energizing portion that deforms the elastic body and increases energizing force toward the distal end direction; and a release portion that releases a deformation state of the elastic body with the energizing force increased by the energizing portion. In the wire feeding device, the wire gripped by the grip portion is fed toward the distal end direction by moving the grip portion in the distal end direction by the energizing force of the elastic body whose deformation state is released by the release portion, and the wire feeding device further includes a grip operation portion that is able to operate a state of grip of the wire by the grip portion.

In the wire feeding device described above, the release portion includes a deformation maintaining portion that is able to maintain the deformation state of the elastic body that is in a predetermined state due to an increase in the energizing force by the energizing portion, and a maintaining state change portion that is able to change a state to a state in which maintaining of the deformation state of the elastic body by the deformation maintaining portion is disabled. In the wire feeding device, the grip operation portion may be able to operate the grip portion gripping the wire to a state of grip release when the deformation state of the elastic body is maintained by the deformation maintaining portion.

The wire feeding device described above may further include a hitting portion that is arranged on a proximal end side of the grip portion, and is movable in an axial direction of the wire and is able to come into contact with and separate from the grip portion.

In the wire feeding device described above, the grip portion may include a first clamping portion and a second clamping portion that clamp the wire, and a cam portion that is able to adjust an interval between the first clamping portion and the second clamping portion.

The wire feeding device described above may further include a placement portion on which the wire to be gripped by the grip portion can be placed, the placement portion having the entire surface in a predetermined direction is opened over a longitudinal direction of the wire to be placed, and an open and close portion that is able to open and close at least a part of the opened surface in the predetermined direction of the placement portion.

The wire feeding device described above may further include an energizing operation portion that is manually operated by a technician in order to increase the energizing force to the elastic body by the energizing portion, in which the energizing operation portion may include a handle portion that is manually operated by the technician at the time of operation to a position offset so as to separate from a placement position of the wire.

### BRIEF DESCRIPTION OF THE DRAWINGS

FIG. 1 is a diagram for explaining a guide wire and a catheter connected to a wire feeding device, and a connector for connecting to the wire feeding device.

FIG. 2 is a diagram for illustrating a connection state between the guide wire and the catheter and the connector.

FIG. 3 is a perspective view of the wire feeding device according to a first embodiment.

FIG. 4 is a perspective view of the wire feeding device to which the guide wire and the catheter are connected.

FIG. 5 is a sectional top view of the wire feeding device illustrated in FIG. 3 in an initial state.

FIG. 6 is a configuration diagram of a grip portion of the wire feeding device.

FIG. 7 is a perspective sectional view from a bottom surface in the initial state illustrated in FIG. 5.

FIG. 8 is a perspective view of an injection switch of the wire feeding device.

FIG. 9 is a perspective view of a slider of the wire feeding device.

FIG. 10 is a perspective view of a hook of the wire feeding device.

FIG. 11 is a sectional top view of the wire feeding device in a gripping preparation state.

FIG. 12 is a sectional top view of the wire feeding device in a feedable state.

FIG. 13 is a perspective sectional view from a bottom surface in the feedable state illustrated in FIG. 12.

FIG. 14 is a sectional bottom view of the wire feeding device at the time of feeding.

FIG. 15 is a sectional top view at the time of feeding illustrated in FIG. 14.

FIG. 16 is a sectional side view of a grip open and close switch of the wire feeding device at the time of non-operation.

FIG. 17 is a sectional top view of the grip open and close switch illustrated in FIG. 16 at the time of operation.

FIG. 18 is a sectional side view at the time operation illustrated in FIG. 17.

FIG. 19 is a sectional side view of the grip open and close switch at the time of non-operation according to a modification.

FIG. 20 is a sectional side view of the grip open and close switch illustrated in FIG. 19 at the time of operation.

FIG. 21 is a sectional top view of the wire feeding device in an initial state according to a second embodiment.

FIG. 22 is a perspective sectional view from a bottom surface in the initial state illustrated in FIG. 21.

FIG. 23 is a perspective view of an injection switch of the wire feeding device.

FIG. 24 is a first perspective view of a slider of the wire feeding device.

FIG. 25 is a second perspective view of the slider illustrate in FIG. 24.

FIG. 26 is a sectional top view of the wire feeding device in a feedable state.

FIG. 27 is a perspective sectional view from a bottom surface in the feedable state illustrated in FIG. 26.

FIG. 28 is a bottom perspective view of the wire feeding device at the time of feeding.

FIG. 29 is a sectional side view of the wire feeding device after feeding.

FIG. 30 is a sectional top view of the wire feeding device in an initial state according to a third embodiment.

FIG. 31 is a sectional side view in the initial state illustrated in FIG. 30.

FIG. 32 is an exploded perspective view of a grip portion of the wire feeding device illustrated in FIG. 30.

FIG. 33 is a perspective view of the grip portion illustrated in FIG. 32 in a gripping release state.

FIG. 34 is a sectional side view in the gripping release state illustrated in FIG. 33.

FIG. 35 is a perspective view of the grip portion in a gripping state.

FIG. 36 is a sectional side view of the grip portion in the gripping state.

FIG. 37 is a perspective view of a grip open and close switch of the wire feeding device illustrated in FIG. 30.

FIG. 38 is a sectional top view of the wire feeding device in a feedable state.

FIG. 39 is a sectional side view in the feedable state illustrated in FIG. 38.

FIG. 40 is an exploded perspective view of a grip portion according to a modification.

FIG. 41 is a perspective view of the grip portion illustrated in FIG. 32 in a gripping release state.

FIG. 42 is a sectional top view of the wire feeding device at the time of lid opening according to a fourth embodiment.

FIG. 43 is a sectional top view at the time of lid opening illustrated in FIG. 42.

FIG. 44 is a top view at the time of lid closing.

FIG. 45 is a perspective view of a lid.

FIG. 46 is an enlarged perspective view of attachment part of the lid.

FIG. 47 is a top view illustrating a state where the guide wire is placed on the wire feeding device.

FIG. 48 is a sectional side view of a part including a guide wire accommodating portion of the wire feeding device.

FIG. 49 is a perspective view of the wire feeding device in a feedable state of the guide wire.

FIG. 50 is a perspective view of a wire feeding device according to a fifth embodiment.

FIG. 51 is a perspective sectional view of the wire feeding device illustrated in FIG. 50 from a bottom surface at the time of non-fixation of an injection switch.

FIG. 52 is a perspective sectional view from the bottom surface at the time of fixation of the injection switch illustrated in FIG. 51.

### DETAILED DESCRIPTION

Hereinafter, the wire feeding device according to embodiments will be described with reference to the drawings. However, the disclosed embodiments are not limited to the embodiments illustrated in the drawings.

In this specification, the term “guide wire” indicates a medical guide wire that is pushed to a surgical site in a body cavity such as a blood vessel and is used to guide a catheter to the surgical site.

In this specification, the “distal end side” and the “distal end direction” indicate the side and direction, along the longitudinal direction of the guide wire (direction along the axial direction of the guide wire), where an occluding object to be penetrated by a guide wire is positioned. The “proximal end side” (i.e., rear end side) and the “proximal end direction” (i.e., rear end direction) are opposite to the distal end side and direction. Moreover, the “proximal end side” indicates a direction along the longitudinal direction of the guide wire, the direction being opposite to the distal end side. Moreover, the “distal end” refers to an end on the distal end side of an arbitrary member or portion, and the “proximal end” refers to an end on the proximal end side of an arbitrary member or portion.

### First Embodiment

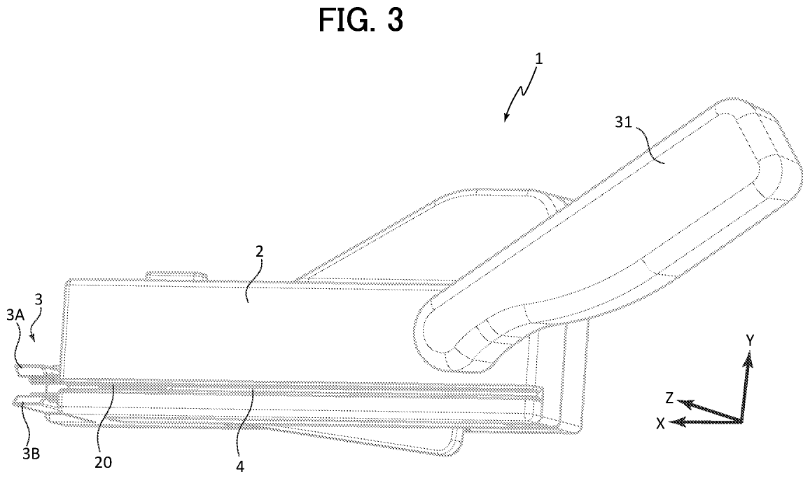

A wire feeding device 1 (see FIG. 3) according to a first embodiment is a device that feeds a guide wire as an example of the wire. The guide wire is pushed to a surgical site in a body cavity such as a blood vessel, for example, and is used to penetrate an occluding object in the surgical site, for example. The wire feeding device 1 is used by connecting a catheter through which the guide wire GW is inserted to the device.

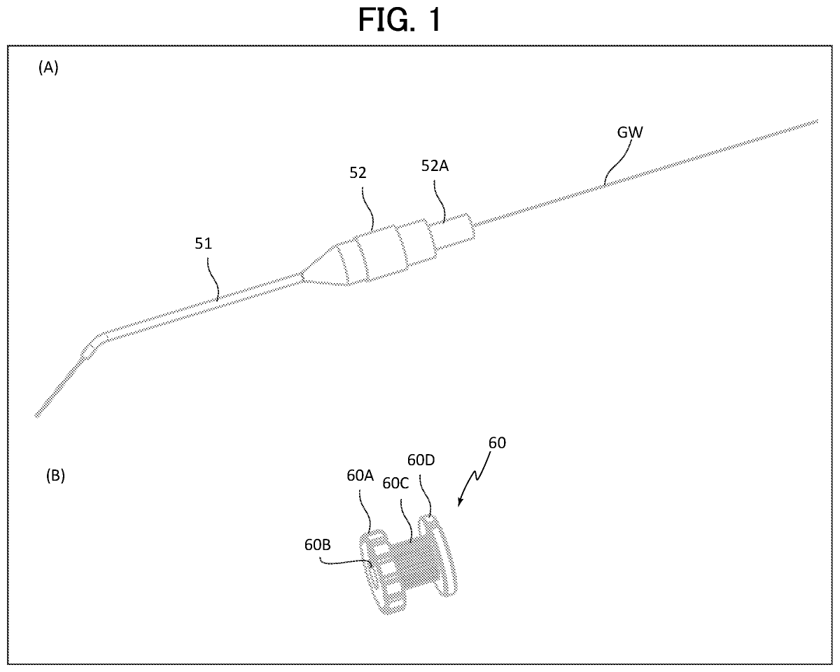

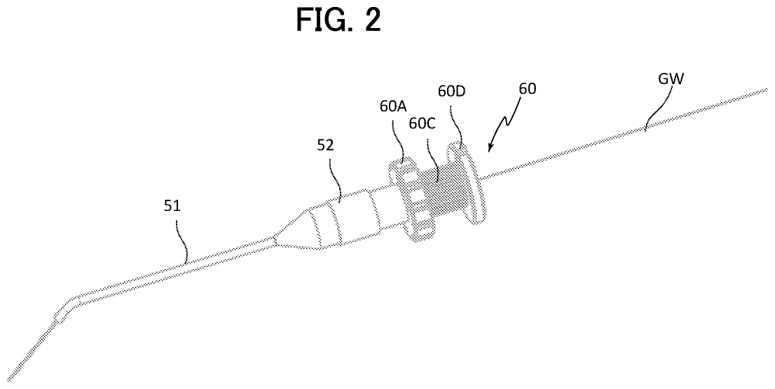

Before describing the details of the wire feeding device 1, a guide wire and a catheter connected to the wire feeding device 1 will be described. FIG. 1 is a diagram for explaining a guide wire and a catheter connected to a wire feeding device 1 and a connector for connecting to the wire feeding device. FIG. 2 is a diagram illustrating a connection state between the guide wire and the catheter and the connector.

The guide wire GW is inserted through a hollow catheter 51. A catheter hub 52 for adjusting the direction of the catheter 51 is non-rotatably attached to proximal end side of the catheter 51. In the example of FIG. 1(A), the left side of the drawing is the inside of the patient's body (distal end side), and the right side of the drawing is the outside of the patient's body (proximal end side).

The catheter 51 is connected to a connector 60 as illustrated in FIG. 2, and is connected to the wire feeding device 1 through the connector 60. As illustrated in FIG. 1(B), the connector 60 includes a dial portion 60A, an attachment portion 60C, and a proximal end portion 60D. The dial portion 60A is a portion for a technician to operate the direction of the catheter 51 connected to the connector 60. The attachment portion 60C is a portion formed in a cylindrical shape for the attachment to a connector connection portion 3 (see FIG. 3) described later of the wire feeding device 1. The length in the axial direction of the attachment portion 60C is substantially the same as the width in the X-axis direction of connecting pieces 3A and 3B described later of the connector connection portion 3. The proximal end portion 60D is formed in a disk shape having a diameter larger than that of the cylinder of the attachment portion 60C. The proximal end portion 60D acts to position the connector 60 in the X-axis direction with respect to the connector connection portion 3.

The connector 60 has a through hole 60B extending in the longitudinal direction. The through hole 60B is configured to engage with a proximal end portion 52A of the catheter hub 52. Engagement of the proximal end portion 52A of the catheter hub 52 with the through hole 60B make it possible for the catheter hub 52 and the connector 60 to integrally rotate.

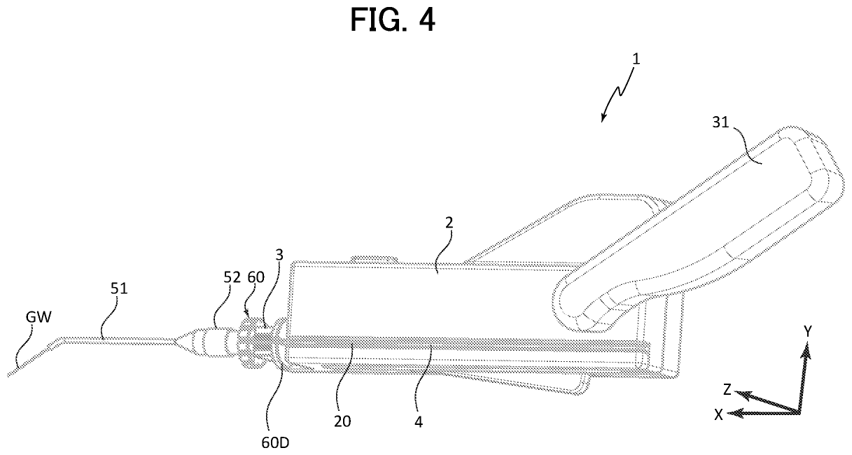

FIG. 3 is a perspective view of the wire feeding device, and FIG. 4 is a perspective view of the wire feeding device to which a guide wire and a catheter are connected. The wire feeding device 1 includes a housing 2, a lever 31, the connector connection portion 3, a guide wire accommodating portion 4, and a grip portion 20.

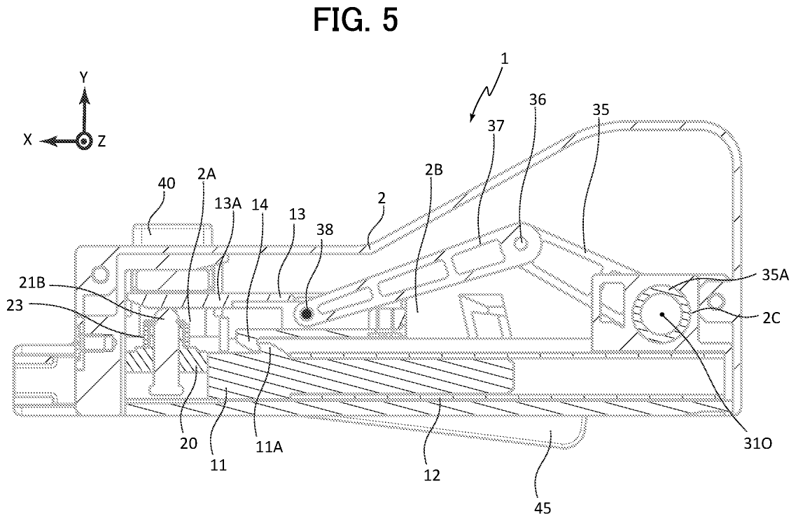

The housing 2 has a substantially rectangular parallelepiped shape with the axial direction (X-axial direction in the drawing) being the longitudinal direction when the guide wire GW is attached. The housing 2 includes therein various components described later for gripping and feeding the guide wire GW in addition to the grip portion 20. The lever 31 is turnable with a lever rotation axis 310 (see FIG. 5) described later as a center, and is a portion operated by a technician to feed the guide wire GW (a first predetermined operation portion). In the present embodiment, the technician can feed the guide wire GW by gripping and rotating the lever 31 with one hand.

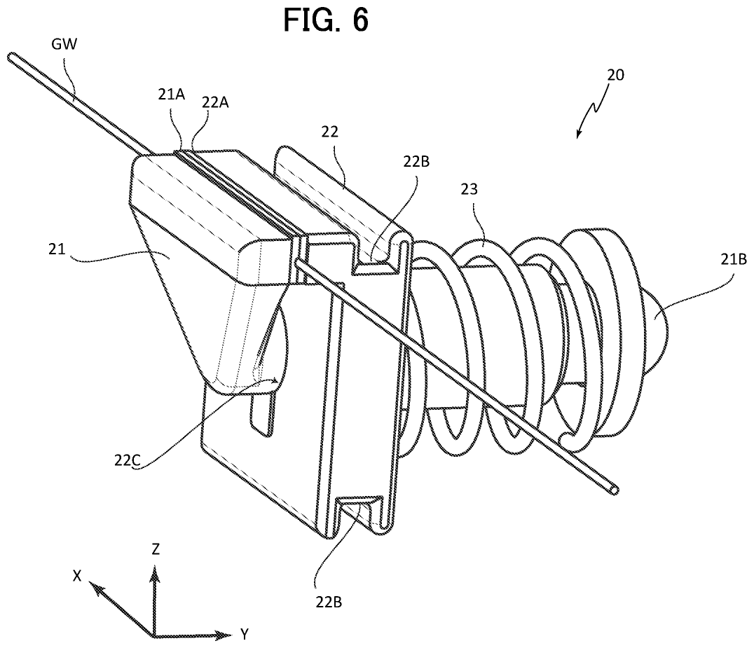

The connector connection portion 3 is a portion for connecting the attachment portion 60C, and includes a pair of connecting pieces 3A and 3B extending in the X-axis direction. The connection pieces 3A and 3B are formed of, for example, elastic bodies such as resin, and sandwich the outer peripheral surface of the attachment portion 60C from both sides in the Y-axis direction to rotatably connect the connector 60. The guide wire accommodating portion 4 is a portion for accommodating the guide wire GW to be fed, and is formed in a concave shape extending in the X-axis direction and being open in the Z-axis positive direction over the entire X-axis direction. The grip portion 20 is a portion that can grip the guide wire GW and can move the guide wire GW in the X-axis direction, and is arranged in the middle part in the X-axis direction of the guide wire accommodating portion 4 such that grip surface portions 21A and 22A (see FIG. 6) described later are open to the outside. In the present embodiment, when the guide wire GW is placed on the surface in the Z-axis negative direction of the guide wire accommodating portion 4 (also referred to here as a bottom surface), the guide wire GW is arranged in space (arrangement space) between the grip surfaces holding the guide wire GW of the grip surface portion 21A and the grip surface portion 22A.

To connect the catheter 51 and the guide wire GW to the wire feeding device 1, the guide wire GW at a part on the more proximal end side than the connector 60 is placed on the bottom surface of the concave portion of the guide wire accommodating portion 4, and then the attachment portion 60C connected to the catheter 51 to which the guide wire GW is inserted is fitted between the connection pieces 3A and 3B for attachment. FIG. 4 illustrates the state in which the catheter 51 and the guide wire GW are connected to the wire feeding device 1 in this manner. In the state where the catheter 51 and the guide wire GW are connected to the wire feeding device 1 in this manner, the direction of the catheter 51 can be easily adjusted by the technician rotating the operation dial 60A (see FIG. 1). In this state, a gap is secured between the surface on the X-axis negative direction side (a direction opposite to the direction of the arrow on the X-axis) of the proximal end portion 60D and the surface on the X-axis positive direction side of the housing 2. Thus, liquid such as blood and chemicals that have passed through the catheter 51 easily flow down from the gap, which prevents the components of the housing 2 from coming into contact with the liquid. Further, when the lever 31 is in an initial state, the guide wire GW is not gripped as described later. Thus, it is possible to adjust the position and direction of the guide wire GW by moving forward and rearward and rotating the guide wire GW.

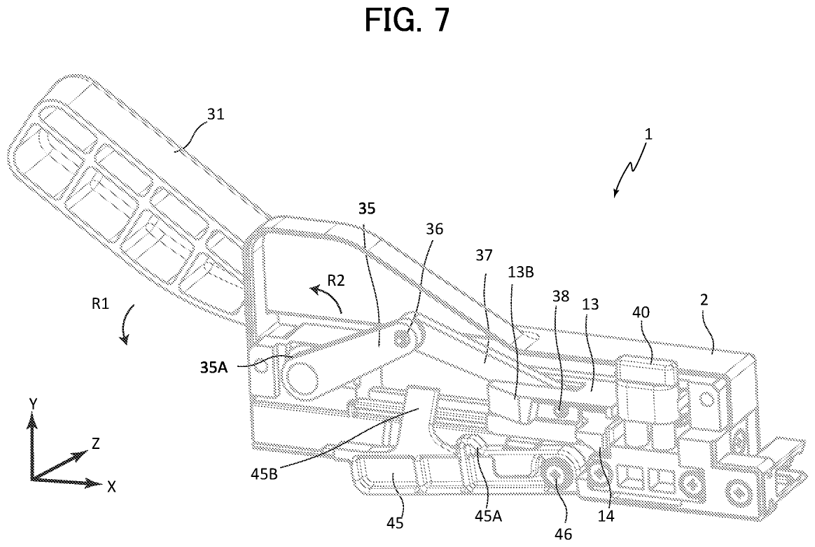

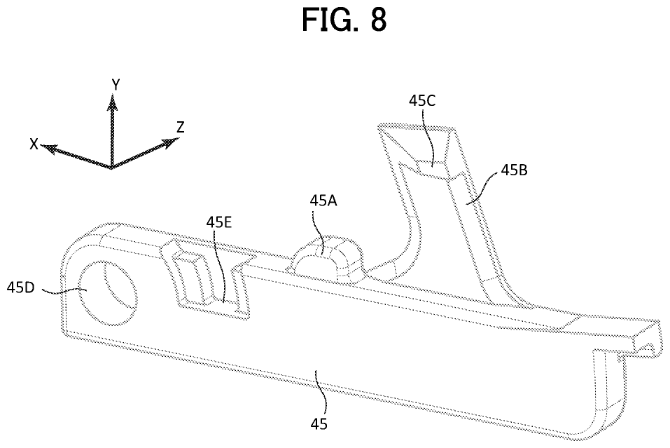

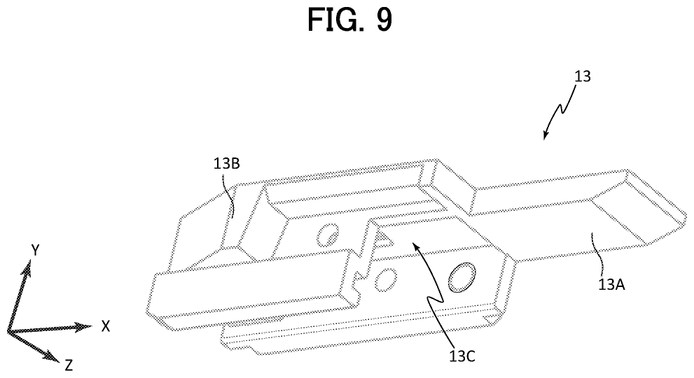

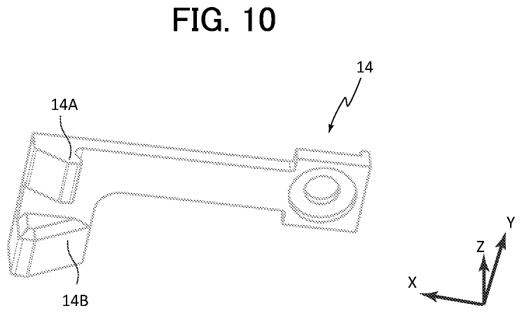

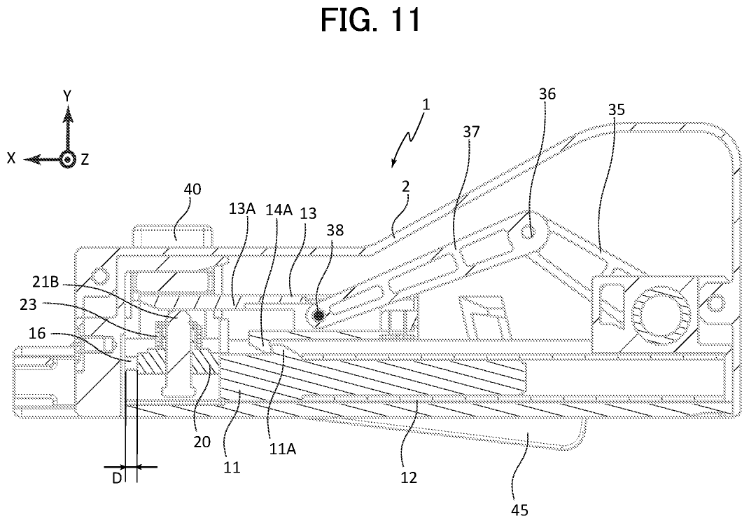

Next, the wire feeding device 1 will be described in detail. FIG. 5 is a sectional top view of the wire feeding device 1 in the initial state. The wire feeding device 1 includes a housing, a grip portion, a push spring, a return spring (see FIG. 11), a slider, a hook, a grip open and close switch, and an injection switch. FIG. 6 is a configuration diagram of the grip portion. FIG. 7 is a bottom perspective view of the wire feeding device 1 in the initial state. FIG. 8 is a perspective view of the injection switch, FIG. 9 is a perspective view of the slider, and FIG. 10 is a perspective view of the hook. FIG. 7 illustrates a state in which a part of the housing in a negative direction side of the Z-axis from the wire feeding device 1 and some other bottom perspective views in other figures may illustrate a similar state in this specification.

The wire feeding device 1 includes a housing 2, a grip portion 20, a push spring 12, a return spring 16 (see FIG. 11), a slider 13, a hook 14, a grip open and close switch 40, and an injection switch 45. As illustrate in FIG. 5, the wire feeding device 1 further preferably includes a hammer 11. The push spring 12 is an example of an elastic body. The slider 13 and the hook 14 are examples of an energizing portion. The hammer 11 is an example of a hitting portion. As illustrated in FIG. 7, the wire feeding device 1 further includes the lever 31, links 35 and 37, and joints 36, 38. The lever 31, the links 35, 37, the joints 36, 38, and the slider 13 are examples of the power transmission mechanism, the slider 13, the hook 14, and the injection switch 45 are examples of the deformation maintaining portion, and the injection switch 45 is an example of the maintaining state change portion and the operation portion and also an example of the second predetermined operation portion. The grip open and close switch 40 is an example of the gripping operation portion. The slider 13, the hook 14, and the injection switch 45 are an example of the release portion.

With the guide wire GW attached, the housing 2 has a substantially rectangular parallelepiped shape extending in the axial direction (X-axial direction in the drawing). Further, as illustrated in FIG. 5, the housing 2 includes a grip portion accommodating portion 2A for accommodating the grip portion 20 movably in the X-axis direction and a slider accommodating portion 2B for accommodating the slider 13 and the hook 14 movably in the X-axis direction. The housing 2 further is formed with a support hole 2C that rotatably supports a cylindrical portion 35A on one end side of the link 35.

The grip portion 20 capable of gripping the guide wire GW is movable in the X-axis direction in the grip portion accommodating portion 2A. As illustrated in FIG. 6, the grip portion 20 includes a first component 21, a second component 22, and a grip spring 23. The first component 21 includes, for example, the grip surface portion 21A extending in the X-axis direction and a substantially columnar leg portion 21B extending in the Y-axis direction. The second component 22 is formed to extend in the X-axis direction and includes the grip surface portion 22A having a surface facing the grip surface portion 21A of the first component 21, a concaves portion 22B having a concave portion facing in both directions in the Z-axis direction, and a through hole portion 22C with a through hole through which the leg portion 21B can be inserted.

The first component 21 and the second component 22 are combined such that the leg portion 21B is inserted in the through hole of the through hole portion 22C. The grip spring 23 is attached such that an energizing force (biasing force) is generated between the leg portion 21B and the second component 22, and the energizing force of the grip spring 23 acts so that the surface (grip surface) on the second component 22 side of the grip surface portion 21A of the first component 21 and the surface (grip surface) on the first component 21 side of the grip surface portion 22A of the second component 22 approach to each other.

In the present embodiment, space (arrangement space) is formed between the grip surface on the second component 22 side of the grip surface portion 21A and the grip surface on the first component 21 side of the grip surface portion 22A. It is possible to arrange the guide wire GW in the arrangement space and for the grip portion 20 to grip the guide wire GW by the energizing force of the grip spring 23. This arrangement space can be opened to the outside by an opening extending over the entire movable direction of the grip portion 20. In this embodiment, the guide wire GW is placed on the concave portion of the guide wire accommodating portion 4, whereby it is possible to position the guide wire GW substantially in the middle in the Z-axis direction of the grip surface of the grip surface portion 21A and the grip surface of the grip surface portion 22A. Meanwhile, when the leg portion 21B is pushed toward the first component 21 side (Y-axis negative direction side) and the grip spring 23 is compressed, the grip surface of the grip surface portion 21A and the grip surface of the grip surface portion 22A are separated from each other, and space is generated therebetween. As a result, the grip portion 20 no longer grips the guide wire GW (grip release).

The concave portion 22B engages with a convex portion (not illustrated) extending in the X-axis direction on both wall portions in the Z-axis direction of the grip portion accommodating portion 2A of the housing 2, and acts to accurately guide the movement of the grip portion 20 in the X-axis direction.

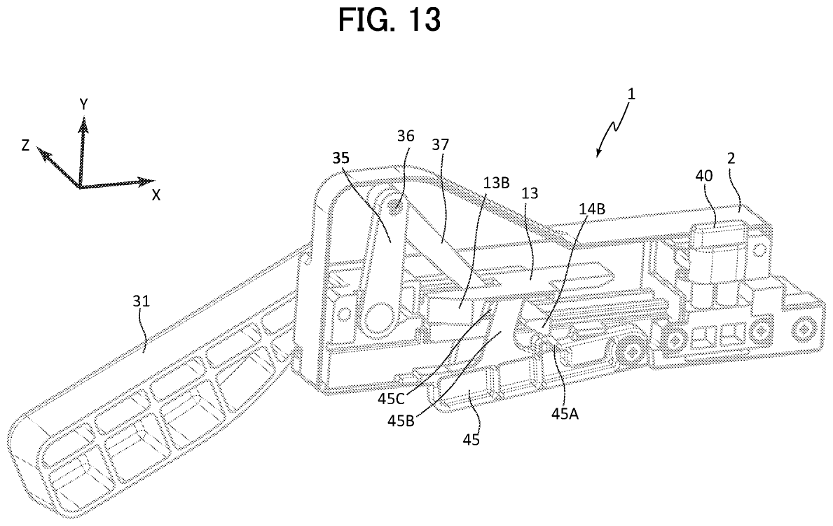

As illustrated in FIG. 8, the injection switch 45 includes a convex portion an extension piece 45B, a convex portion 45C, a fixation hole 45D, and a spring accommodating portion 45E. With rotating the injection switch 45 the convex portion 45A contacts the convex portion 14B (see FIGS. 10 and 13) of the hook 14 and thereby, the convex portion 45A is able to push up the convex portion 14B. The extension piece 45B is formed extending in a positive direction of the Y-axis. The convex portion 45C is formed in a distal end of the extension piece in the Y-axis positive direction and protrudes in a Z-axis negative direction. The convex portion 45C engages with the convex portion 13B (see FIGS. 9 and 13) of the slider 13 and is able to limit the movement of the slider 13 in the X-axis direction. The fixation hole 45D is a hole to which a screw 46 for fixing the injection switch 45 rotatably to the housing 2. The injection switch 45 is fixed to the housing 2 by the screw 46 and thereby, the injection switch 45 can rotate around the fixation hole 45D. The spring accommodating portion 45E accommodates a spring (not illustrated) that energizes the injection switch 45 in the Y-axis negative direction. The injection switch 45 itself may be configured to be elastic so that the injection switch 45 is energized in the Y-axis negative direction without using a spring.

As illustrated in FIG. 9, the slider 13 includes an extension portion 13A, a convex portion 13B capable of engaging with the extension piece 45B and the convex portion 45C, and an attachment portion 13C capable of attaching the hook 14. The extension portion 13A has a plate-shape in which the surface in the Y-axis negative direction on the distal end side in the X-axis direction is thinner toward the distal end side. As illustrated in FIG. 5, when the distal end portion of the extension portion 13A is moved to the distal end side of the X-axis direction, the extension portion 13 is in a state of pushing a leg portion 21B of the grip portion 20 to the Y-axis negative direction side, compresses the grip spring 23, and moves the leg portion 21B in the Y-axis negative direction. As a result, the surface on the second component 22 side of the grip surface portion 21A and the surface on the first component 21 side of the grip surface portion 22A are separated from each other, thereby releasing the grip of the guide wire GW.

The hook 14 is attached to the attachment portion 13 of the slider. As illustrated in FIG. 10, the hook 14 includes a spring hook 14A and a convex portion 14B. The spring hook 14A is able to engage with the convex portion 11A of the hammer 11. The convex portion 14B is a portion that contacts the convex portion 45A of the injection switch 45. When the convex portion 14B is pushed in the Y-axis positive direction by the convex portion 45A, the hook 14 elastically deforms and the spring hook 14A is moved in the same direction. In the present embodiment, the slider 13 and the hook 14 are separately configured, but may be integrally configured.

As illustrated in FIG. 5, the hammer 11 is arranged on the proximal end side in the X-axis direction of the grip portion 20 such that the longitudinal direction of the hammer 11 is the X-axis direction. The push spring 12 is arranged around a part on the proximal end side of the hammer 11 and on the proximal end side of the hammer 11 such that the longitudinal direction of the push spring 12 is the X-axis direction.

The hammer 11 is made of metal, for example, and is movable in the X-axis direction. The hammer 11 includes a convex portion 11A on the side of the slider accommodating portion 2B. The convex portion 11A can be engaged with the spring hook 14A. The push spring 12 is, for example, a metal spring, and is deformable (compressible) in the X-axis direction and capable of applying energizing force in the X-axis positive direction on the hammer 11.

The return spring 16 is, for example, a metal spring, and is deformable (compressible) in the X-axis direction and energizes the grip portion 20 toward the proximal end side. The energizing force of the return spring 16 that is applied on the grip portion 20 is smaller than the energizing force of the push spring 12 in the initial state that is applied on grip portion 20 (a state in which the compression is not generated by the movement of the hammer 11). In this manner, when the push spring 12 is in the initial state, the grip portion 20 is positioned at the most distal end position (i.e., the frontmost end position) in the movable range (the frontmost end position in the X-axis direction in the grip portion accommodating portion 2A). Meanwhile, when the push spring 12 is compressed so that no energizing force is applied on the grip portion 20, the grip portion 20 is moved to the rearmost end position (i.e., the most proximal end position) in the movable range (the rearmost end position in the X-axis direction in the grip portion accommodating portion 2A) by the energizing force of the return spring 16.

In the wire feeding device 1, the lever 31, the links 35, 37, the joints 36, 38, and the slider 13 form the power transmission mechanism.

The lever 31 is a portion for a technician using the wire feeding device 1 to manually perform a turning operation. The lever 31 is turnable around the lever rotation axis 310. As illustrated in FIG. 7, the lever 31 and the cylindrical portion 35A of the link 35 are connected through the joint (not illustrated) such that the turning force is transmittable from the lever 31 to the link 35. In the present embodiment, the link 35 is configured to turn integrally with the turn of the lever 31.

The other end of the link 35 and one end of the link 37 are rotatably connected through the joint 36. The other end of the link 37 and the slider 13 are rotatably connected through the joint 38. The slider 13 is linearly movable in the X-axis direction.

In this power transmission mechanism, when the lever 31 is turned in an R1 direction, the link 35 is turned in an R2 direction. With the turn of the link 35, the link 37 moves with the movement of the slider 13 along the X-axis direction. In the present embodiment, the turn angle of the lever 31, the lengths of the links 35 and 37, and the like of this power transmission mechanism are adjusted such that in a turnable range of the lever 31, the slider 13 can move in the entire moving range in the X-axis direction.

When the slider 13 moves from the frontmost end position to the proximal end side in the X-axis direction in the moving range of the slider 13, the spring hook 14A engages with the convex portion 11A of the hammer 11. When the slider 13 further moves, the hammer 11 is moved to the proximal end side, so that the push spring 12 is compressed. When the slider 13 approaches the rearmost end position, the convex portion 45C of the extension piece 45B of the injection switch climbs over the convex portion 13B of the slider 13 in the X-axis positive direction so that the convex portion 45C and the convex portion 13B engage with each other. As a result, the slider 13 is disable to move in the X-axis positive direction in a state where the push spring 12 is compressed. Thereafter, when the injection switch 45 is pushed in the Y-axis positive direction, the convex portion pushes the convex portion 14B of the hook 14 in the Y-axis positive direction, the portion of the hook 14 in the X-axis positive direction side is deformed in the Y-axis positive direction, and engagement between the spring hook 14A of the hook 14 and the convex portion 11A of the hammer 11 is released. As a result, the deformation state (compression state) of the push spring 12 is released at once and the push spring 12 pushes the hammer 11 in the X-axis direction.

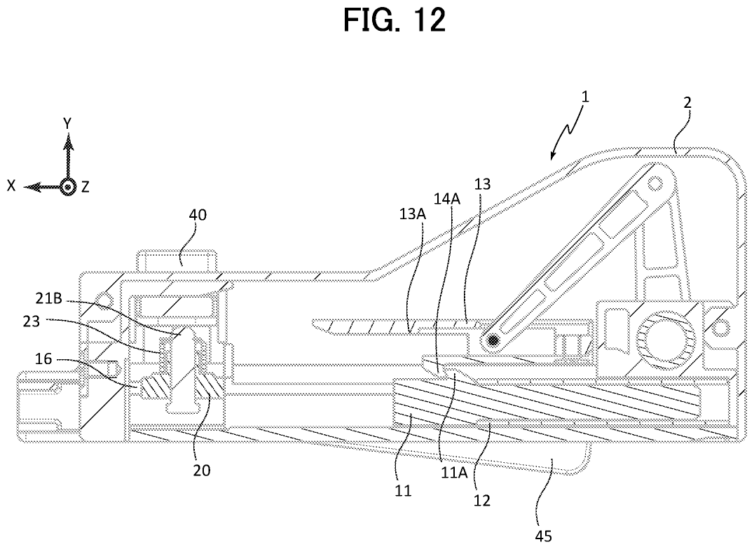

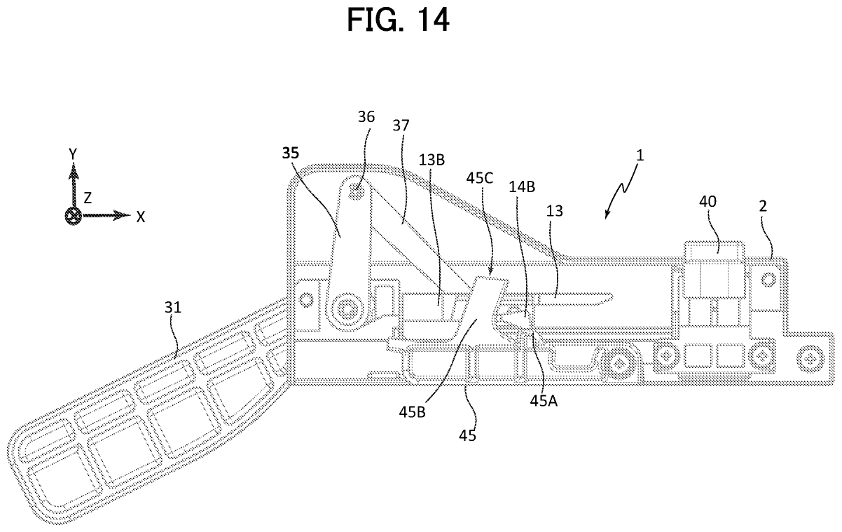

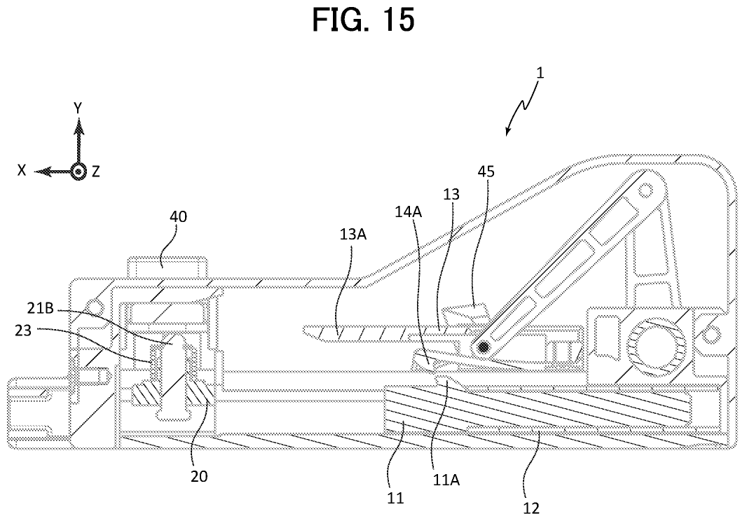

The following will specifically describe a use method of the wire feeding device 1 and the operation of the wire feeding device 1, with reference to the drawings. FIG. 11 is a sectional top view of the wire feeding device in a gripping preparation state, FIGS. 12 and 13 are a sectional top view and a bottom perspective view in a feedable state, and FIGS. 14 and 15 are a sectional bottom view of the wire feeding device and a sectional top view at the time of feeding. In the wire feeding device 1, the grip portion 20, the hammer 11, the slider 13, and the hook 14 are configured to interlock with each other so that (a) gripping the guide wire GW, (b) moving the grip portion 20 toward the distal end direction of the guide wire GW, (c) releasing the grip of the guide wire GW, and moving the grip portion 20 toward the proximal end direction are performed in this order.

First, a technician inserts the guide wire GW into a blood vessel, and then pushes the guide wire GW to an obstructed site along the blood vessel. Next, after the distal end of the guide wire GW reaches the obstructed site, a catheter 51 is pushed to an obstructed site using the guide wire GW as a guide. Then, the connector 60 is connected to the catheter hub 52 of the catheter 51, while the connector 60 is pushed into the connector connection portion 3 from the Z-axis positive direction, the proximal end side of the guide wire GW is accommodated in the guide wire accommodating portion 4 of the housing 2 from the Z-axis positive direction side, so that the connector 60 is connected to the wire feeding device 1.

In this case, as illustrated in FIG. 5, the extension portion 13A pushes the leg portion 21B in the Y-axis negative direction, and the grip spring 23 is compressed. Thus, the surface on the second component 22 side of the grip surface portion 21A and the surface on the first component 21 side of the grip surface portion 22A are separated from each other, and arrangement space is formed therebetween, in the state where the grip of the guide wire GW by the grip portion 20 is released. Therefore, as described above, the catheter 51 with the guide wire GW inserted therethrough is attached to the wire feeding device 1, thereby making it possible for the guide wire GW to be easily accommodated in the arrangement space. This can reduce the preparation time for feeding the guide wire GW by the wire feeding device 1, and reduces loads on the patient and the technician.

Next, turning of the lever 31 slightly in the R1 direction of FIG. 7 slides the slider 13 and the hook 14 toward the proximal end side. Since the convex portion 11A of the hammer 11 is engaged with the spring hook 14A, as illustrated in FIG. 11, the hammer 11 is moved toward the proximal end side with the movement of the slider 13, so that the push spring 12 is compressed.

Here, the grip portion 20 is no longer pushed toward the X-axis positive direction by the hammer 11. Thus, the grip portion 20 slides to the rearmost end in the movable range by the energizing force of the return spring 16. As a result, as illustrate in FIG. 11, the grip portion 20 moves by a distance D from the frontmost end (initial position) to the rearmost end in the movable range. The distance D corresponds to a feeding amount per time by the wire feeding device 1. For example, when the feeding amount per time by the wire feeding device 1 is 2 mm, the grip portion 20 slides to the proximal end side by 2 mm from the initial position. Here, the extension portion 13A is configured to be at a position where the grip spring 23 of the grip portion 20 is compressed, which keeps the state where the grip portion 20 does not grip the guide wire GW.

Further, when the lever 31 is turned in the R1 direction, the slider 13 and the hook 14 further slide toward the proximal end side. Since a state in which the spring hook 14A is engaged with the convex portion 11A of the hammer 11 is maintained, the hammer 11 is moved toward the proximal end side with the movement of the slider 13 and the hook 14, so that the push spring 12 is further compressed.

At this time, the extension portion 13A does not contact the leg portion 21B and does not compress the grip spring 23 anymore, so that the guide wire GW is gripped by the surface on the second component 22 side of the grip surface portion 21A and the surface on the first component 21 side of the grip surface portion 22A.

When the lever 31 is rotated to the position at which the slider 13 is on the most proximal end side of the moving range (feedable position), the slider 13 and the hook 14 further slide to the proximal end side, and as illustrated in FIGS. 12 and 13, the convex portion 45C of the injection switch 45 climbs over the convex portion 13B of the slider 13 and located in the X-axis positive direction of the convex portion 13B, so that the convex portion 45C and the convex portion 13B engage with each other. As a result, the slider 13 is disabled to move in the X-axis positive direction, the state in which the push spring 12 is compressed is maintained, and a state in which feeding is enabled (compression maintaining state: feedable state) is established.

Thereafter, when the injection switch 45 is pressed in the positive direction of the Y-axis by the technician, as illustrated in FIG. 14, the convex portion 45A presses the convex portion 14B in the positive direction of the Y-axis, the portion of the hook 14 in the X-axis positive direction side is bent in the Y-axis positive direction, the spring hook 14A moves in the Y-axis positive direction as illustrated in FIG. 15, and a state in which the spring hook 14A is engaged with the convex portion 11A of the hammer 11 is released. The convex portion 45C of the extension piece 45B moves in the positive direction further than the surface of the slider 13 in the Y-axis positive direction and does not engage with the convex portion 13B anymore, so that the slider 13 is enabled to move in the X-axis positive direction.

In this manner, the energizing force of the push spring 12 is applied at once to the movement of the hammer 11 toward the distal end direction, so that the hammer 11 moves toward the distal end direction, and the distal end side of the hammer 11 hits the proximal end side of the grip portion 20.

As a result, the grip portion 20 gripping the guide wire GW moves toward the distal end direction by the impact due to a hit with the hammer 11, and stops at the frontmost end position of the grip portion 20. Here, the extension portion 13A of the slider 13 is not in contact with the grip portion 20, thereby keeping the state of gripping the guide wire GW.

Therefore, the grip portion 20 moves from the rearmost end position to the frontmost end position while keeping the state of gripping the guide wire GW. As a result, the guide wire GW is fed to the distal end side by the distance D from the rearmost end position to the frontmost end position of the grip portion 20.

Thereafter, when the technician rotates the lever 31 in a direction opposite to R1, the slider 13 and the hook 14 moves to the X-axis positive direction, and the wire feeding device 1 can be return to the initial state as illustrated in FIG. 5. The operations up to this correspond to a single mode (second mode) of feeding the wire one time. In the single mode, after (a) the grip portion 20 grips the guide wire GW and the slider 13 and the hook 14 deform the push spring 12, the injection switch 45 is pressed, so that (b) the grip portion 20 gripping the guide wire GW is fed to the distal end side by release of the deformation and energizing of the push spring 12, and then (c) the gripping is released and feeding of the guide wire GW is enabled.

When the guide wire GW need to be continuously fed, it is sufficient that the lever 31 is continuously rotated and similar operations are performed (first mode). Maintaining the state in which the injection switch 45 is pressed makes it possible for the wire feeding device 1 to be operated in a continuous mode. When the continuous mode is set by maintaining the state in which the injection switch 45 is pressed, the technician operates the lever 31, and the push spring 12 is compressed, the convex portion 45A of the injection switch 45 pushes the convex portion 14B, so that the spring hook 14A of the hook 14 is detached from the convex portion 11A of the hammer 11 and the hammer 11 hits the grip portion 220. As a result, the guide wire GW can be fed in the distal end direction. Thereafter, when the technician continuously operates the lever 31, the guide wire GW can be continuously fed to the distal end side (first mode). That is, a series of operations can be repeated in the order of above-described (a), (b), and (c).

As described above, in the wire feeding device 1, it is possible to feed the guide wire GW by only an appropriate amount by applying impact force due to the energizing force accumulated in the push spring 12 on the guide wire GW. In this manner, it is possible to apply impact force on the guide wire GW, which allows the guide wire GW to penetrate an occluding object more effectively.

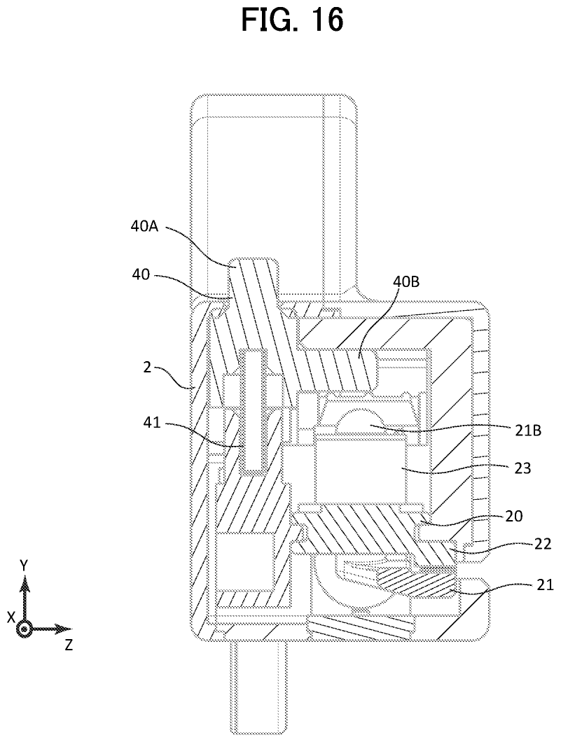

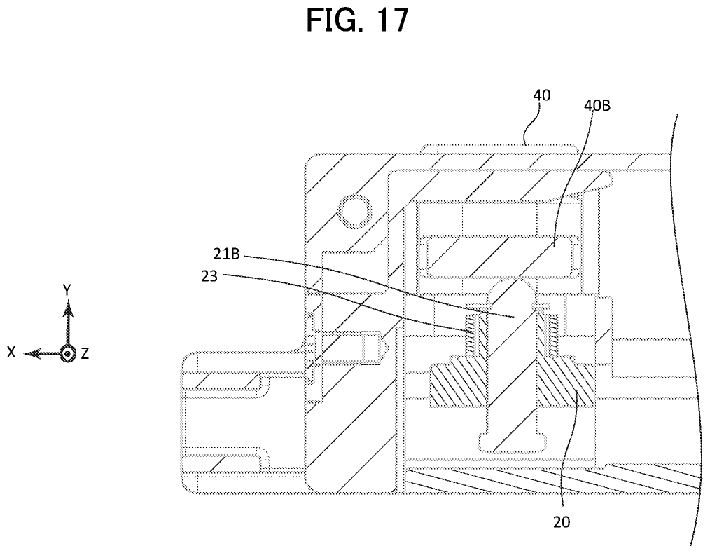

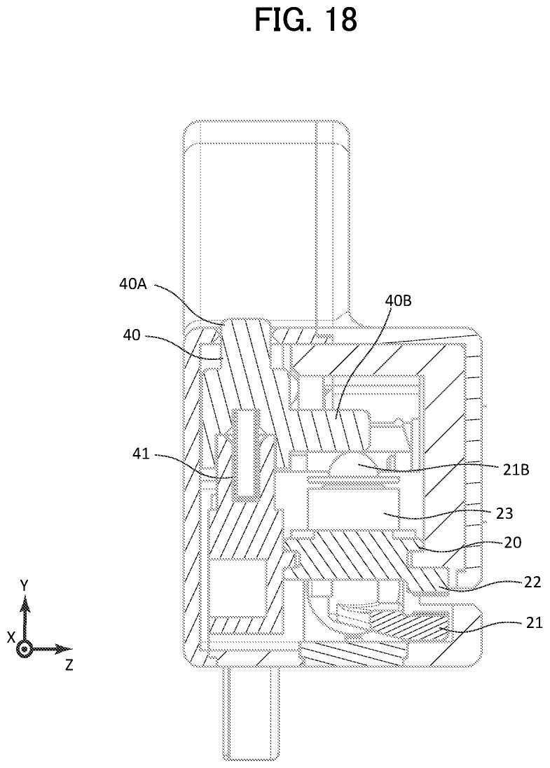

Next, the configuration and operation of peripheral portions of the grip open and close switch 40 for releasing the grip of the guide wire GW gripped by the grip portion 20. FIG. 16 is a sectional side view at the time of non-operation of the grip open and close switch of the wire feeding device, and FIGS. 17 and 18 are a sectional top view and sectional side view at the time of operation of the grip open and close switch.

The grip open and close switch 40 has a L-shape cross-sectional shape as illustrated in FIG. 16, and includes a pressing portion 40A that is pressed by a technician, and an action portion 40B that is arranged in the Y-axis positive direction with respect to the leg portion 21B of the grip portion 20, and acts so as to push the leg portion 21B in the Y-axis negative direction when the pressing portion 40A is pushed. The grip open and close switch 40 is movable in the Y-axis direction. The grip open and close switch 40 is energized in the Y-axis positive direction by the spring 41, and is configured such that, when the technician is not pushing the pressing portion 40A, as illustrated in FIG. 16, the action portion 40B does not contact the leg portion 21B. In the present embodiment, the grip open and close switch 40 is adjusted such that the action portion 40B (see FIG. 16) is located so as not to contact the extension portion 13A of the slider 13 when the technician is not pushing the pressing portion 40A, as illustrated in FIG. 11.

In the state illustrated in FIG. 16, since the grip spring 23 of the grip portion 20 is not being compressed, the guide wire GW is gripped in between the surface on the second component 22 side of the grip surface portion 21A of the first component 21 and the surface on the first component 21 of the grip surface portion 22A of the second component 22.

For example, in a case where the guide wire feeding device 1 is in the feedable state as illustrated in FIGS. 12 and 13, when the grip open and close switch 40 (pressing portion 40A) is pushed in the Y-axis negative direction as illustrated in FIGS. 17 and 18, the action portion 40B pushes the leg portion 21B in the Y-axis negative direction, and the grip spring 23 is compressed. Therefore, the surface on the second component 22 side of the grip surface portion 21A and the surface on the first component 21 side of the grip surface portion 22A are separated from each other and grip of the guide wire GW by the grip portion 20 is released.

Thus, according to the wire feeding device 1, pushing the grip open and close switch 40 makes it possible to release grip of the guide wire GW by the grip portion 20, even when the wire feeding device 1 is in the feedable state as illustrated in FIG. 12 for example. Therefore, after the feedable state is established, releasing the grip of the guide wire GW by the grip portion 20 makes it possible to easily perform adjustment such as changing a position and direction of the guide wire GW.

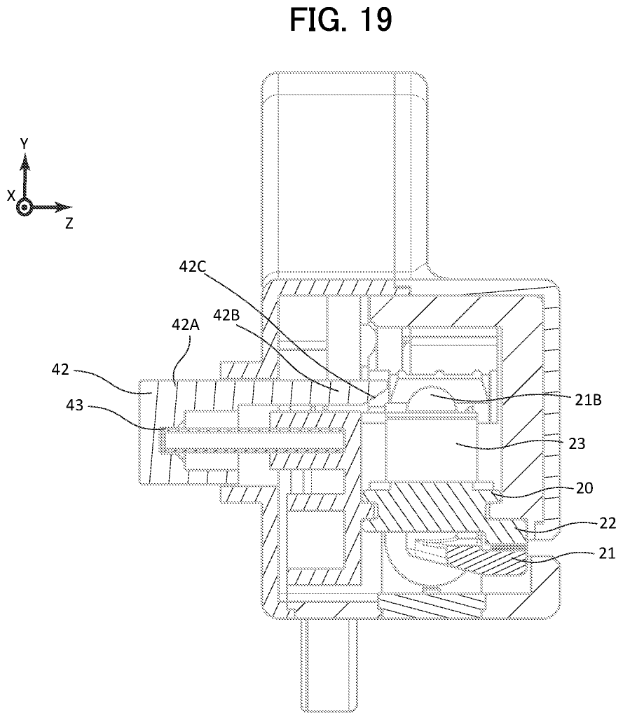

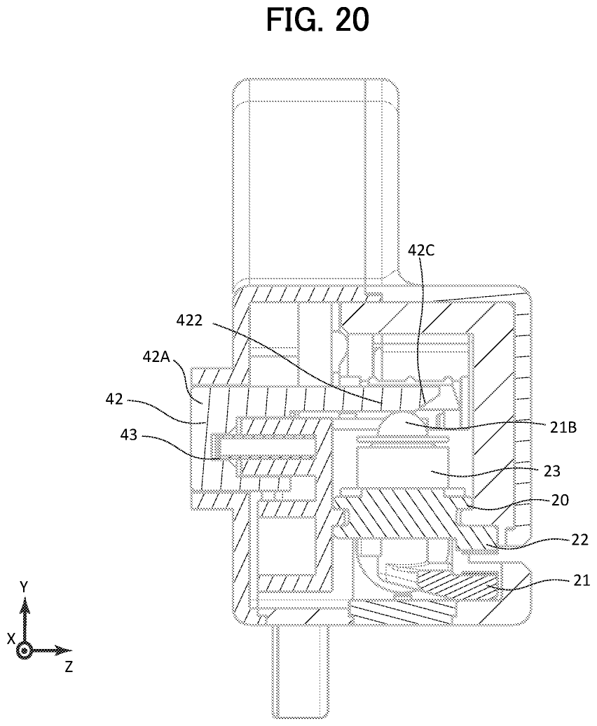

Next, the configuration and operation according to a modification for releasing the grip of the guide wire GW gripped by the grip portion 20. This modification is a grip open and close switch in which the grip by the grip portion 20 can be released by pushing the grip open and close switch in the Z-axis direction. FIG. 19 is a sectional side view of the grip open and close switch at the time of non-operation according to the modification, and FIG. 20 is a sectional side view at the time of operation.

As illustrated in FIG. 19, the grip open and close switch 42 according to the modification includes a pressing portion 42A that is pressed by a technician, and an action portion 42B that is arranged in the Z-axis negative direction with respect to the leg portion 21B of the grip portion 20, and acts so as to push the leg portion 21B in the Y-axis negative direction when the pressing portion 42A is pushed. The surface of the action portion 42B in the Y-axis negative direction in the Z-axis positive direction end portion is an inclined surface 42C that is further in the Y-axis negative direction toward the Z-axis negative direction. Therefore, when the action portion 42B is pushed in the Z-axis positive direction, the inclined surface 42C abuts the leg portion 21B and the leg portion 21B is pushed in the Y-axis negative direction. The grip open and close switch 42 is movable in the Z-axis direction. The grip open and close switch 42 is energized in the Z-axis negative direction by the spring 43, and is configured such that, when the technician is not pushing the pressing portion 42A, the inclined surface 42C of the action portion 42B does not contact the leg portion 21B.

In the state illustrated in FIG. 19, since the grip spring 23 of the grip portion 20 is not being compressed, the guide wire GW is gripped in between the surface on the second component 22 side of the grip surface portion 21A of the first component 21 and the surface on the first component 21 of the grip surface portion 22A of the second component 22.

For example, in a case where the guide wire feeding device 1 is in the feedable state, as illustrated in FIG. 20, when the grip open and close switch 42 (pressing portion 42A) is pushed in the Z-axis positive direction, the inclined surface 42C of the action portion 42B pushes the leg portion 21B in the Y-axis negative direction, and the grip spring 23 is compressed, so that the surface on the second component 22 side of the grip surface portion 21A of the first component 21 and the surface on the first component 21 side of the grip surface portion 22A of the second component 22 are separated from each other, an arrangement space is formed, and grip of the guide wire GW by the grip portion 20 is released.

As described above, pushing the grip open and close switch 42 allows the grip portion 20 to release grip of the guide wire GW by even when the wire feeding device 1 is in the feedable state. Therefore, after the feedable state is established, releasing the grip of the guide wire GW by the grip portion 20 makes it possible to easily perform adjustment such as changing a position and direction of the guide wire GW.

### Second Embodiment

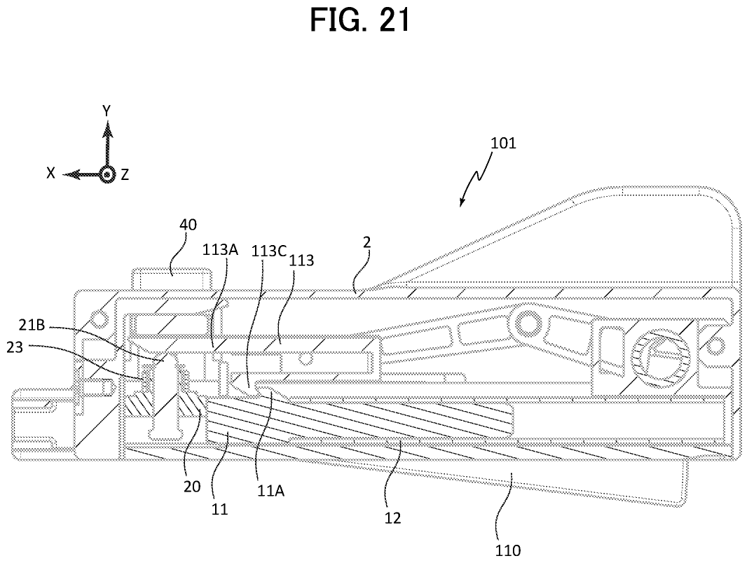

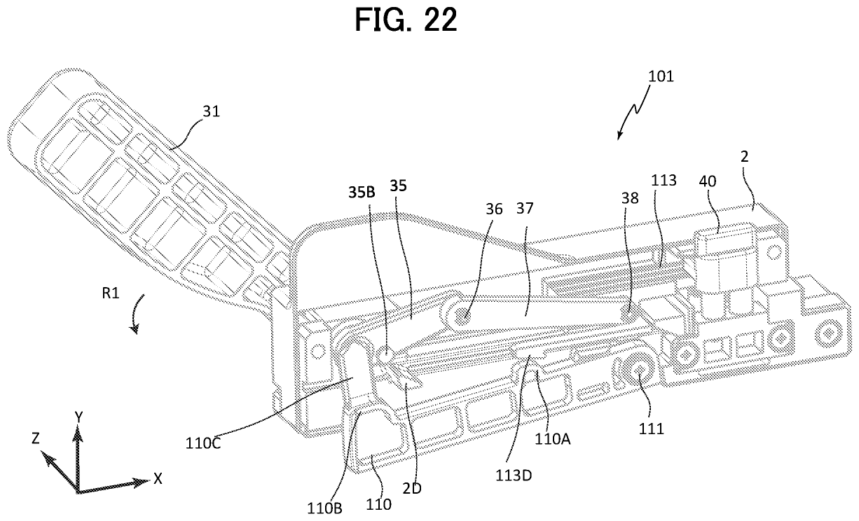

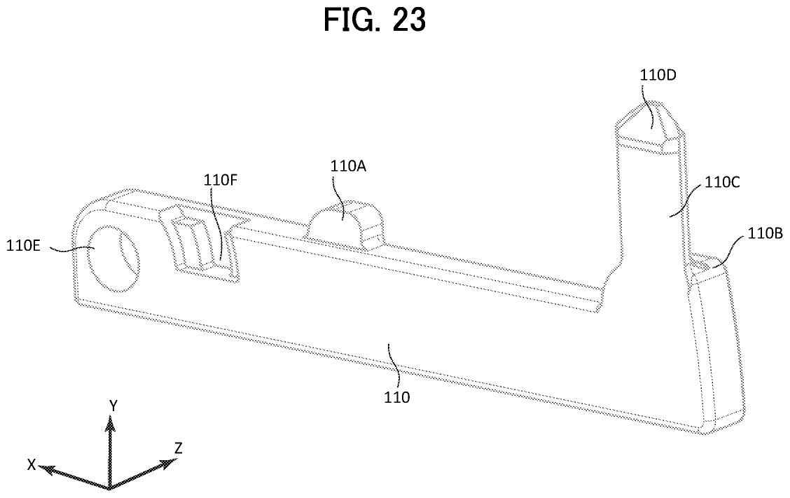

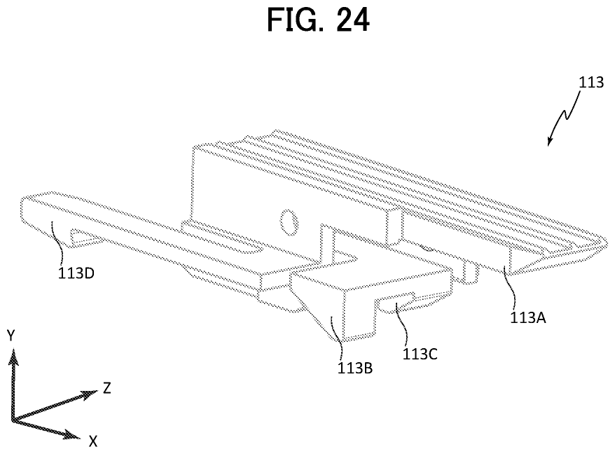

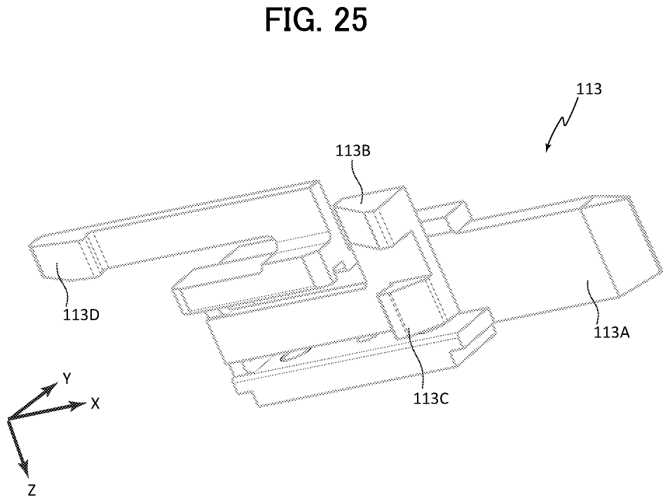

A wire feeding device 101 according to a second embodiment will be described with reference to the drawings. FIGS. 21 and 22 are a sectional top view and a bottom perspective view of the wire feeding device 101 in an initial state, FIG. 23 is a perspective view of an injection switch of the wire feeding device 101, FIG. 24 is a first perspective view of a slider, and FIG. 25 is a second perspective view of the slider. Portions similar to those of the first embodiment are denoted by the same numbers and redundant description is omitted.

The wire feeding device 101 further includes a convex portion 2D (see FIG. 22) in the housing 2, further includes a convex portion 35B in the link 35, includes an injection switch 110 instead of the injection switch 45, and includes a slider 113 instead of the slider 13 and the hook 14.

The convex portion 2D of the housing 2 is a portion capable of engaging with the hook 113D of the slider 113. The convex portion 35B of the link 35 is a portion capable of engaging with the convex portion 110D (see FIG. 23) of the injection switch 110.

As illustrated in FIG. 23, the injection switch 110 includes a convex portion 110A, a convex portion 110B, an extension piece 110C, a convex portion 110D, a fixation hole 110E, and a spring accommodating portion 110F. With rotating the injection switch 110, the convex portion 110A contacts the convex portion 113B (see FIG. 24) of the slider 113 and thereby, the convex portion 110A is able to push up the convex portion 113B. With rotating the injection switch 110 the convex portion 110B contacts the hook 113D of the slider 113 and thereby, the convex portion 110B is able to push up the hook 113D. The extension piece 110C is formed extending in the Y-axis positive direction. The convex portion 110D protrudes in the Z-axis negative direction in a distal end of the extension piece 110C in the Y-axis positive direction. The convex portion 110D engages with the convex portion 35B of the link 35, disturbs engagement between the convex portion 113D and the convex portion 2D, and enable movement of the slider 113 in the X-axis direction. The fixation hole 110E is a hole to which a screw 111 (see FIG. 22) for fixing the injection switch 110 rotatably to the housing 2. The injection switch 110 is fixed to the housing 2 by the screw 111 and thereby, the injection switch 110 can rotate around the rotation hole 110E. The spring accommodating portion 110F accommodates a spring (not illustrated) that energizes the injection switch 110 in the Y-axis negative direction. The injection switch 110 itself may be configured to be elastic so that the injection switch 110 is energized in the negative direction of the Y-axis without including a spring.

As illustrated in FIGS. 24 and 25, the slider 113 includes an extension portion 113A, a convex portion 113B, a spring hook 113C, and a hook 113D. The extension portion 113A extends in the X-axis positive direction and has a plate-shape in which the surface in the negative direction of the Y-axis on the distal end side in the X-axis direction is thinner toward the distal end side. As illustrated in FIG. 21, when the distal end portion of the extension portion 113A is moved to the distal end side of the X-axis direction, the extension portion 113A is in a state of pushing a leg portion 21B to the Y-axis negative direction side, compresses the grip spring 23, and moves the leg portion 21B in the Y-axis negative direction. As a result, the surface on the second component 22 side of the grip surface portion 21A of the first component 21 and the surface on the first component 21 side of the grip surface portion 22A of the second component 22 are separated from each other, thereby releasing the grip of the guide wire GW. The convex portion 113B is a portion that contacts the convex portion 110A of the injection switch 110. The spring hook 113C is able to engage with the convex portion 11A of the hammer 11. When the convex portion 113B is pushed by the convex portion 110A, the spring hook 113C is moved in the same direction. The hook 113D is able to engage with the convex portion 2D of the housing 2. When the hook 113D engages with the convex portion 2D, movement of the slider 113 in the X-axis positive direction is restricted.

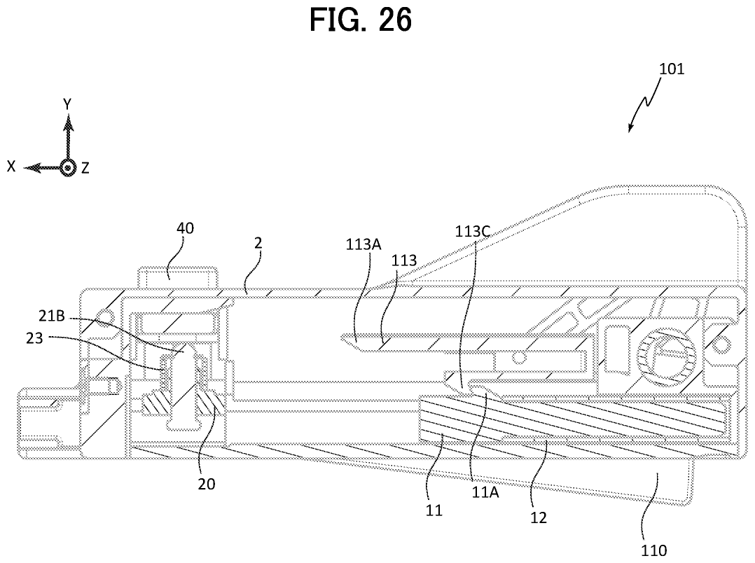

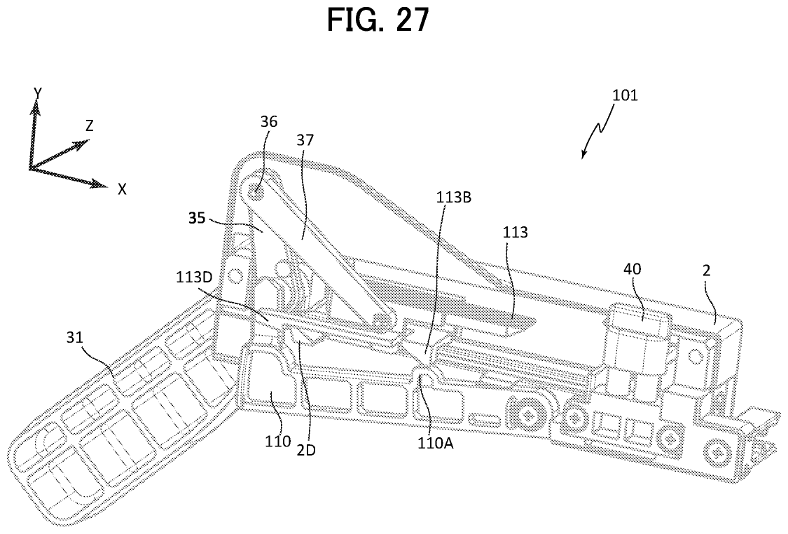

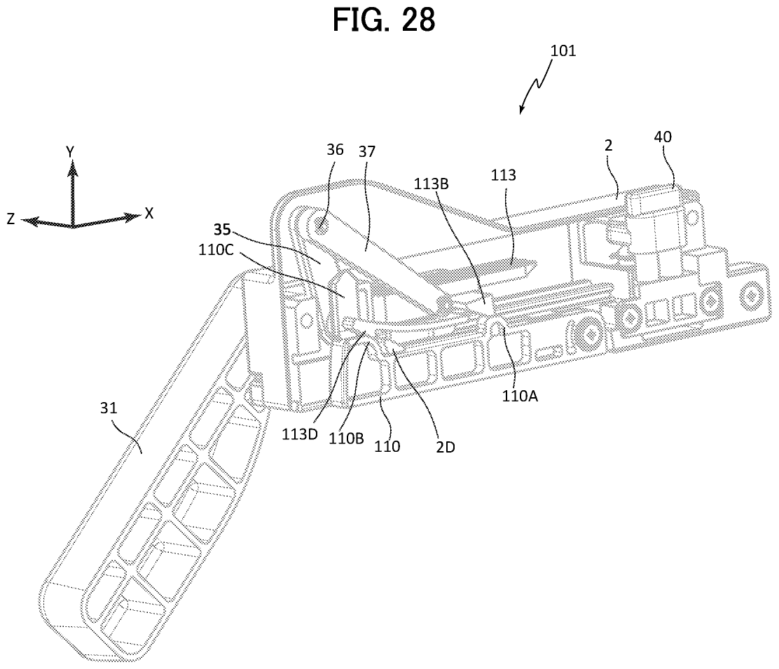

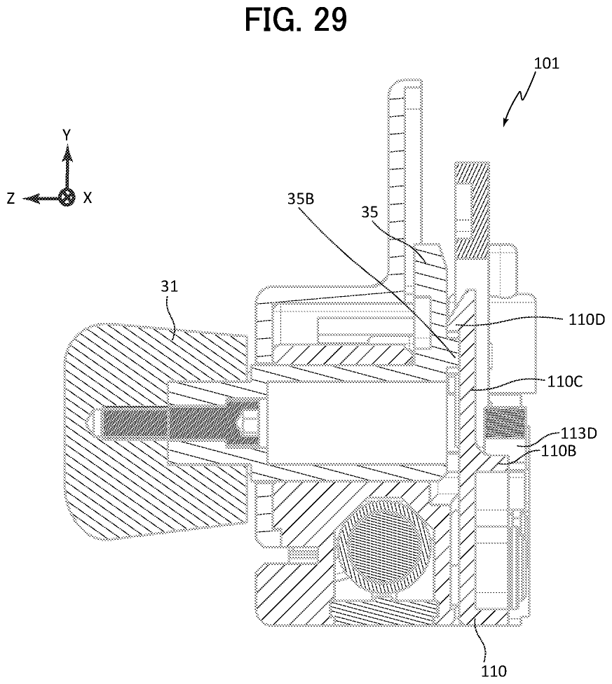

The following will specifically describe a use method of the wire feeding device 101 and the operation of the wire feeding device 101, with reference to the drawings. FIGS. 26 and 27 are a sectional top view and a bottom perspective view of the wire feeding device in a feedable state, FIG. 28 is a bottom perspective view of the wire feeding device at the time of feeding, and FIG. 29 is a sectional side view of the wire feeding device after feeding. In FIGS. 26 to 29, although the guide wire GW, the catheter 51, the connector 60, and the like are not illustrated, subsequent processing will be described assuming that the catheter 51 and the connector 60 are connected to the wire feeding device 101. Portions similar to those in the wire feeding device according to the first embodiment are denoted by the same numbers and redundant description is omitted.

Also in the wire feeding device 101, the grip portion 20, the hammer 11, and the slider 113 are configured to interlock with each other so that gripping the guide wire GW, moving the grip portion 20 toward the distal end direction of the guide wire GW, releasing the grip of the guide wire GW, and moving the grip portion 20 toward the proximal end direction are performed in this order.

In this case, in the wire feeding device 101, as illustrated in FIG. 21, the extension portion 113A pushes the leg portion 21B in the Y-axis negative direction, and the grip spring 23 is compressed. Thus, the surface on the second component 22 side of the grip surface portion 21A of the first component 21 and the surface on the first component 21 side of the grip surface portion 22A of the second component 22 are separated from each other, and arrangement space is formed therebetween, in the state where the grip of the guide wire GW by the grip portion 20 is released.

Next, when the lever 31 is turned in the R1 direction of FIG. 22, the slider 113 slides toward the proximal end side. As illustrated in FIG. 26, since the convex portion 11A of the hammer 11 is engaged with the spring hook 113C of the slider 113, the hammer 11 is moved toward the proximal end side with the movement of the slider 113, so that the push spring 12 is compressed.

Here, the grip portion 20 is no longer pushed toward the X-axis positive direction by the hammer 11. Thus, the grip portion 20 slides to the rearmost end in the movable range by the energizing force of the return spring 16 (see FIG. 11). As a result, the grip portion 20 moves from the frontmost end (initial position) to the rearmost end in the movable range.

Further, when the lever 31 is turned in the R1 direction, the slider 113 further slides toward the proximal end side. Since a state in which the spring hook 113C of the slider 113 is engaged with the convex portion 11A of the hammer 11 is maintained, the hammer 11 is moved toward the proximal end side with the movement of the slider 113, so that the push spring 12 is further compressed.

At this time, the extension portion 113A does not contact the leg portion 21B of the grip portion 20 and does not compress the grip spring 23 anymore, so that the guide wire GW is gripped by the surface on the second component 22 side of the grip surface portion 21A and the surface on the first component 21 side of the grip surface portion 22A.

When the lever 31 is rotated to the position at which the slider 113 is on the most proximal end side of the moving range (feedable position), the slider 113 further slides to the proximal end side, and as illustrated in FIGS. 26 and 27, the hook 113D climbs over the convex portion 2D of the housing 2, so that the hook 113D and the convex portion 2D engage with each other. As a result, the state in which the push spring 12 is compressed is maintained, and a state in which feeding is enabled (compression maintaining state: feedable state) is established and the slider 113 is disabled to move in the X-axis positive direction.

Thereafter, when the injection switch 110 is pushed in the Y-axis positive direction by the technician, as illustrated in FIG. 28, the convex portion 110A pushes the convex portion 113B of the slider 113 in the Y-axis positive direction, and thereby, the spring hook 113C moves in the Y-axis positive direction to be released from a state in which the spring hook 113C is engaged with the convex portion 11A of the hammer 11.

In this manner, the energizing force of the push spring 12 is applied at once to the movement of the hammer 11 toward the distal end direction, so that the hammer 11 moves toward the distal end direction, and the distal end side of the hammer 11 hits the proximal end side of the grip portion 20.

As a result, the grip portion 20 gripping the guide wire GW moves toward the distal end direction by the impact due to a hit with the hammer 11, and stops at the frontmost end position of the grip portion 20. Here, the extension portion 113A of the slider 113 is not in contact with the grip portion 20, thereby keeping the state of gripping the guide wire GW.

Therefore, the grip portion 20 moves from the rearmost (i.e., most proximal) end position to the frontmost (i.e., most distal) end position while keeping the state of gripping the guide wire GW. As a result, the guide wire GW is fed to the distal end side from the rearmost end position to the frontmost end position of the grip portion 20.

Thereafter, when the technician further pushes the injection switch 110 in the Y-axis positive direction, the convex portion 110B pushing the hook 113D in the Y-axis positive direction makes the hook 113D release from a state in which the hook 113D is engaged with the convex portion 2D of the housing 2. And as illustrated in FIG. 29, the convex portion 110D climbs over the convex portion 35B of the link 35 in the Y-axis positive direction. As a result, the injection switch 110 maintains the state in which the convex portion 110D is located further in the Y-axis positive direction than the convex portion 35B. This state maintains the one in which the hook 113D does not engage with the convex portion 2D. That is, the state is maintained in which the slider 113 is movable in the X-axis positive direction.

Thereafter, rotation of the lever 31 by the technician in a direction opposite to the R1 direction can make the slider 113 move to the X-axis positive direction and the wire feeding device 101 return to the initial state as illustrated in FIG. 21. When the guide wire GW need to be continuously fed, it is sufficient that the lever 31 is rotated in the similar manner to that described above and similar operations are performed.

As described above, in the wire feeding device 101 according to the present embodiment, it is possible to feed the guide wire GW by only an appropriate amount by applying impact force due to the energizing force accumulated in the push spring 12 on the guide wire GW. In this manner, it is possible to apply impact force on the guide wire GW, which allows the guide wire GW to penetrate an occluding object more effectively.

### Third Embodiment

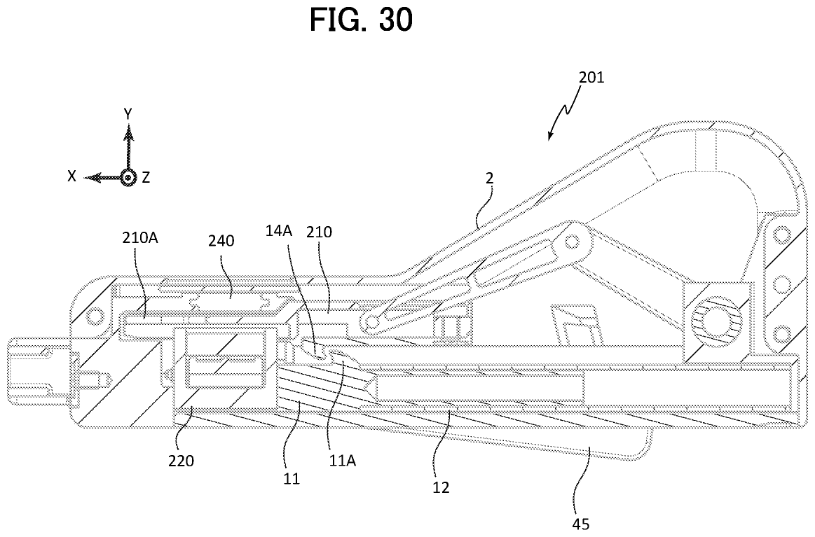

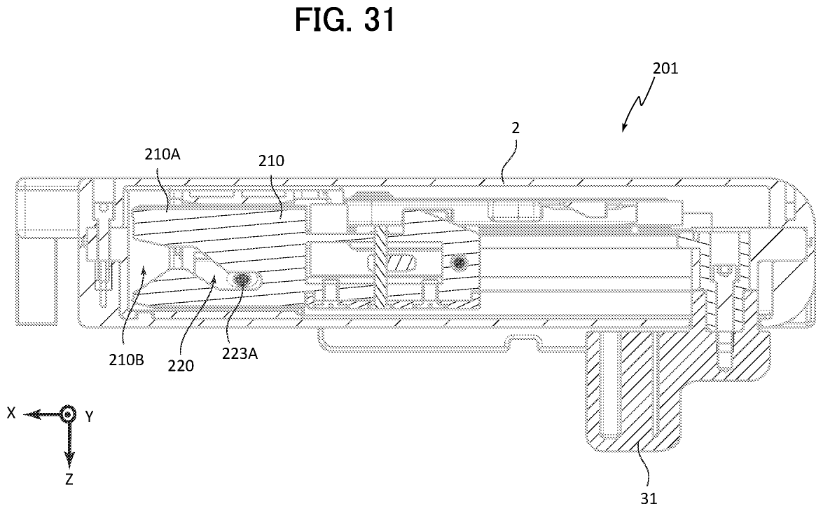

A wire feeding device 201 according to a third embodiment will be described. FIGS. 30 and 31 are a sectional top view and a sectional side view of the wire feeding device in an initial state according to the third embodiment. Portions similar to those of the first and the second embodiments are denoted by the same numbers and redundant description is omitted.

The wire feeding device 201 includes a grip portion 220 instead of the grip portion 20, includes a slider 210 instead of the slider 13 and the hook 14, and includes a grip open and close switch 240 instead of grip open and close such as the grip open and close switch 40, in the wire feeding device 1.

The slider 210 has a configuration in which the slider 13 and the hook 14 are integrated and includes an extension portion 210 instead of the extension portion 13A. The extension portion 210A is formed with a slid 210B that guides an operation convex portion 223A of the grip portion 220 at the time of movement of the slider 210. The slit 210B has an end portion in the X-axis positive direction having a width of equal to or greater than a movable range of the operation convex portion 223A in the Z-axis direction. In the present embodiment, the slit 210B is formed such that, in a case where the slider 210 has moved to the proximal end side, when the operation convex portion 223A is guided to a position (the Z-axis negative direction side) at which the grip portion 220 grips the guide wire GW and the slider 210 moves to the end portion of the distal end side, the operation convex portion 223A is guided to a position (Z-axis positive direction side) at which grip of the guide wire GW by the grip portion 220 is released. The shape of the slit 210B is not limit thereto and may be changed according to a use situation.

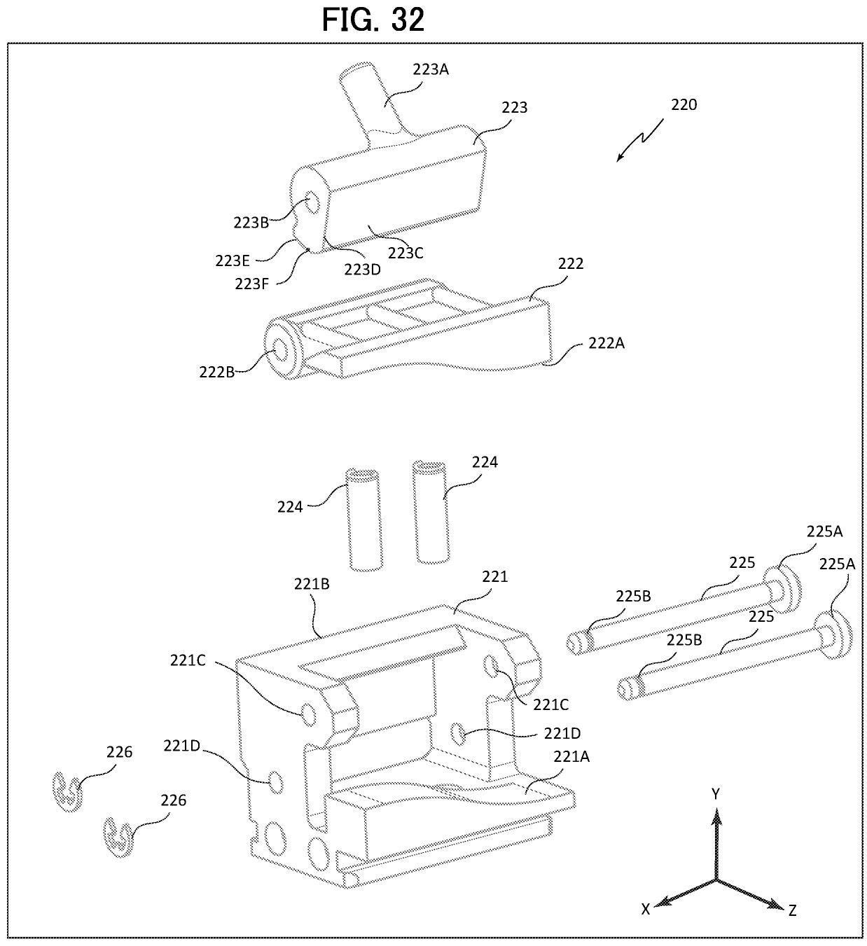

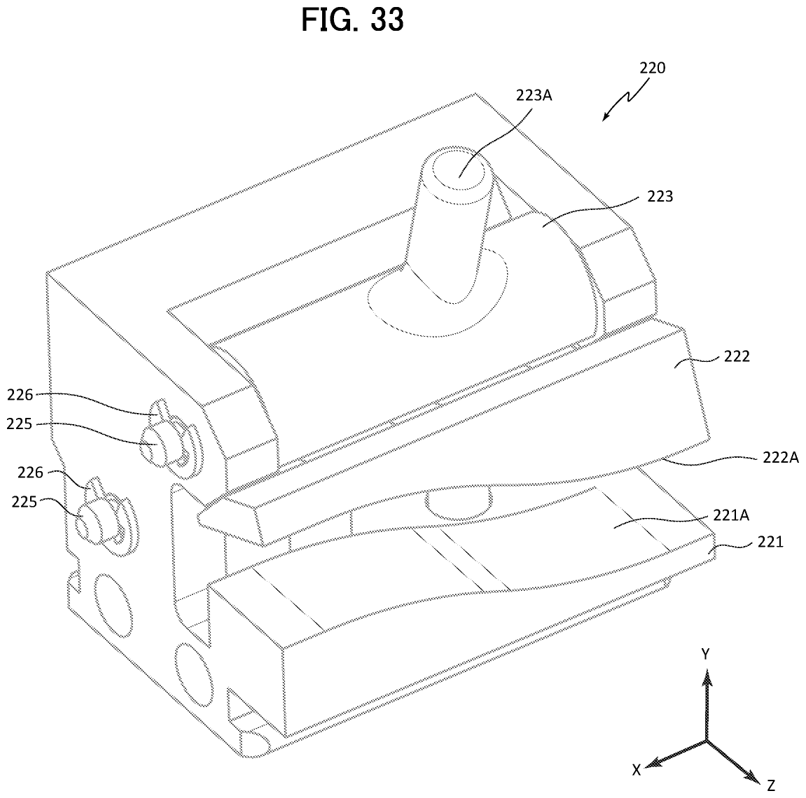

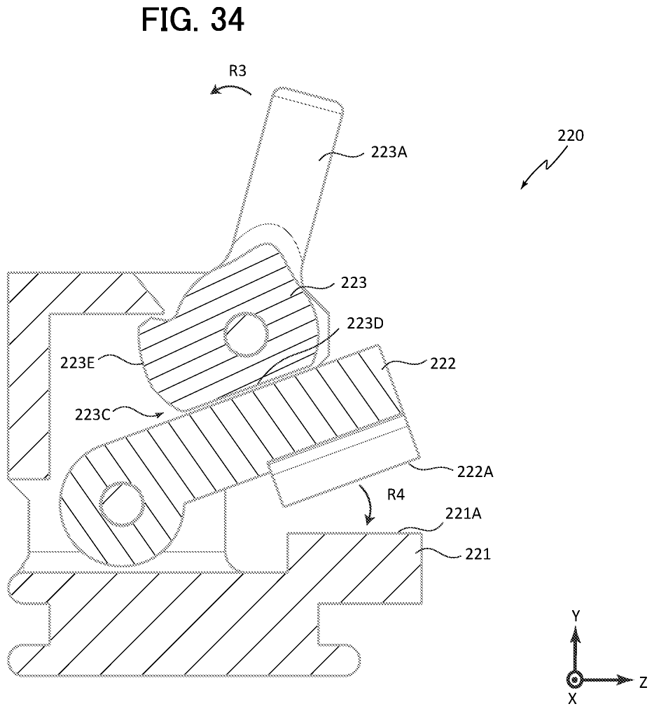

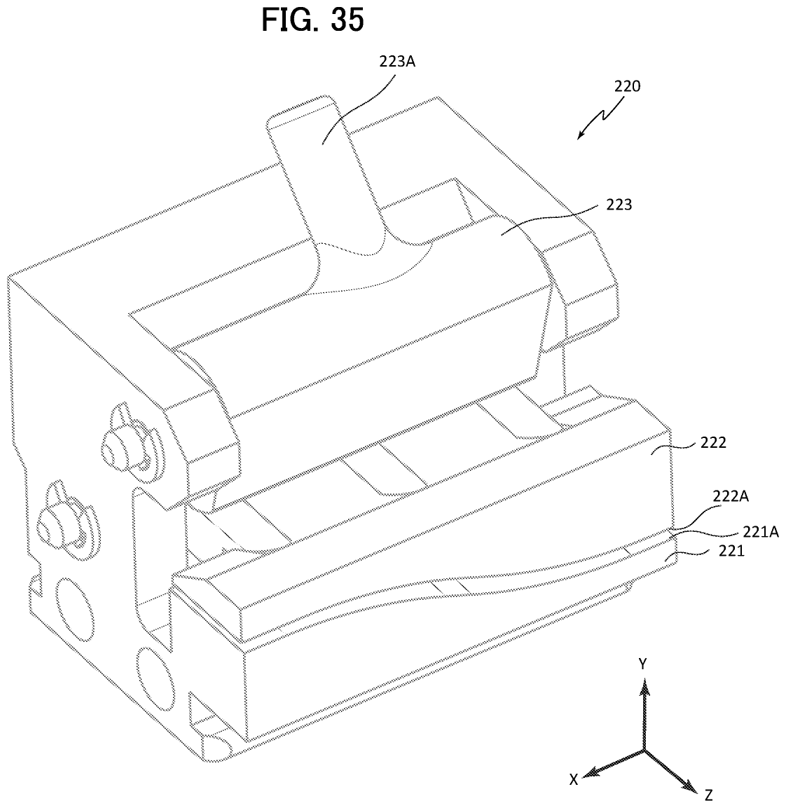

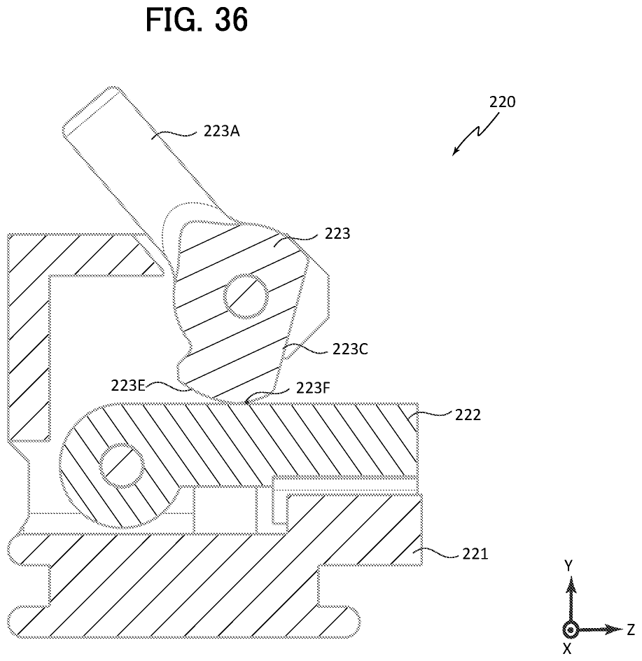

Next, the grip portion 220 will be described. FIG. 32 is an exploded perspective view of the grip portion, FIGS. 33 and 34 are a perspective view and a sectional side view of the grip portion in the grip released state, and FIGS. 35 and 36 are a perspective view and a sectional side view of the grip portion in the grip state.

The grip portion 220 includes a main body portion 221 as an example of a first clamping portion, a facing component 222 as an example of a second clamping portion, a cam portion 223, a grip canceled spring 224, a support pin 225, and a stop ring 226.

The main body portion 221 includes a grip surface 221A and a wall portion 221B. The grip surface 221A is a surface in the Y-axis negative direction that grips the guide wire GW, and for example, a rubber member may be attached to the grip surface 221A. In the present embodiment, the grip surface 221A includes a surface crossing with the grip portion 220 in the movable direction (X-axis direction). The wall portion 221B includes a hole 221C to which a support pin 225 rotatably supporting the cam portion 223 is inserted, and a hole 221D to which a support pin 225 rotatably supporting a facing component 222 is inserted.

The cam portion 223 includes an operation convex portion 223A, a through hole 223B, and a contact surface 223C. The operation convex portion 223A is a portion provided on a side surface of the cam portion 223, for operating the state of the cam portion 223. The through hole 223B is a hole to which the supporting pin 225 for rotating and supporting the cam portion 223 is inserted. The contact surface 223C is a surface that contacts a surface of the facing component 222 in the Y-axis positive direction. A cross-sectional shape (the same shape as the side surface) of a surface perpendicular to the X-axis of the contact surface 223C has a straight portion 223D and a curved portion 223E. At an intermediate point of the curved portion 223E, a maximum contour portion 223F having the maximum distance from a rotating shaft of the cam portion 223. The maximum contour portion 223F is located on the Z-axis positive direction side slightly from the position of the curved portion 223E that the grip portion 220 contacts when the cam portion 223 is in a state in which the grip portion 220 grips the guide wire GW. As a result, the cam portion 223 can be maintained to a state in which the grip portion 220 grips the guide wire GW.

The facing component 222 includes a grip surface 222A and a through hole 222B. The grip surface 222A is a surface in the Y-axis positive direction that grips the guide wire GW, and has a surface shape corresponding to the grip surface 221A of the main body portion 221. The grip surface 222A may be attached with a rubber member. The through hole 222B is a hole to which the supporting pin 225 for rotating and supporting the facing component 222 is inserted.

The grip cancel spring 224 is arranged and energized so as to have a space between facing surfaces of the main body portion 221 and the facing component 222. In the present embodiment, when the cam portion 223 does not fix the grip state by the grip portion 220, specifically, when the cam portion 223 and the facing component 222 contact each other at a Z-axis positive direction position further than the maximum contour portion 223F or when the cam portion 223 and the facing component 222 contact the linear portion 223D, the grip cancel spring 224 acts to have a space between facing surfaces of the main body portion 221 and the facing component 222.

The support pin 225 is a columnar member, includes a head portion 225A in a first end portion, and includes a groove portion 225B to which the stop ring 226 is fit on a second end portion side opposite to the first end portion.

When the stop ring 226 is mounted to the groove portion 225B of the support pin 225, the stop ring 226 prevents the support pin 225 from falling from the holes 221C, 221D.

Next, an assembly method of the grip portion 220 will be described.

First, in a state where the grip cancel spring 224 is assembled in the main body portion 221, the support pin 225 is inserted to the hole 221D of the main body portion 221 in the X-axis negative direction, the through hole 222B of the facing component 222, and a hole 221D of the main body portion 221 in the X-axis positive direction, and the stop ring 226 is mounted to the groove portion 225B of the support pin 225. Next, the support pin 225 is inserted to the hole 221C of the main body portion 221 in the X-axis negative direction, the through hole 223B of the cam portion 223, and a hole 221C of the main body portion 221 in the X-axis positive direction, and the stop ring 226 is mounted to the groove portion 225B of the support pin 225. As a result, the grip portion 220 as illustrated in FIG. 33 is completed.

The grip portion 220 illustrated in FIG. 33 shows a state (grip cancel state) in which grip of the guide wire GW is released. In this state, as illustrated in FIG. 34, in the grip portion 220, the linear portion 223D of the contact surface 223C of the cam portion 223 contacts a surface of the facing component 222 in the Y-axis positive direction, and the grip surface 222A and the grip surface 221A are separated from each other.

On the other hand, when the operation convex portion 223A of the camp portion 223 is rotated in the R3 direction, the curved portion 223E of the contact surface 223C of the cam portion 223 contacts the surface of the facing component 222 in the Y-axis positive direction. As a result, rotation of the grip surface 222A of the facing component 222 in the R4 direction makes a space small between the grip surface 222A and the grip surface 221A as illustrated in FIG. 35 to get the grip surface 222A in touch with the grip surface 221A in the end.

In this state, as illustrated in FIG. 36, the position of the grip portion 220 in the Z-axis negative direction position further than the maximum contour portion 223F contacts a surface of the facing component 222 in the Y-axis positive direction, and the grip surface 222A and the grip surface 221A contact each other. Since the maximum contour portion 223F has a distance from a rotation shaft of the camp portion 223 is longer than that of the position of the contact, the maximum contour portion 223F prevents the cam portion 223 in a direction opposite to the R3 direction, and as a result, is possible to maintain the state in which the grip surface 222A and the grip surface 221A contact each other. This state is released by an application of predetermined force or more in a direction opposite to the R3 direction to the operation convex portion 223A, as illustrated in FIGS. 33 and 34. The grip portion 220 is in a state in which the grip surface 222A and the grip surface 221A are separated from each other, which means a grip released state. A protrusion that prevents the cam portion 223 from moving in a direction opposite to the R3 direction may be provided on an upper surface of the facing component 222 so as to maintain the state in which the grip surface 222A and the grip surface 221A contact each other.

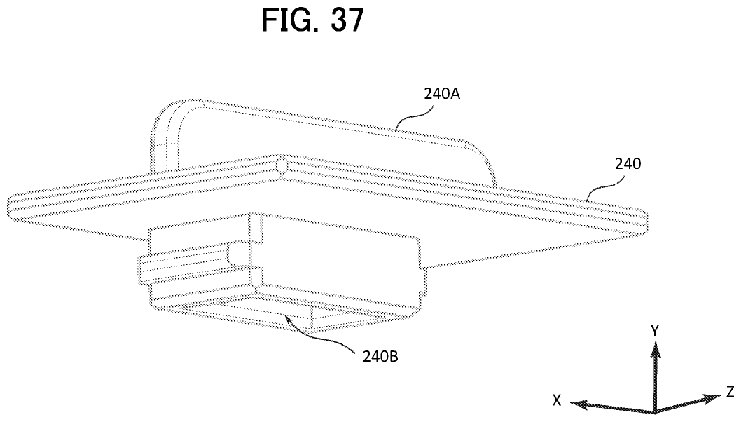

Next, the grip open and close switch 240 will be described. FIG. 37 is a perspective view of a gripping open and close switch of the wire feeding device.

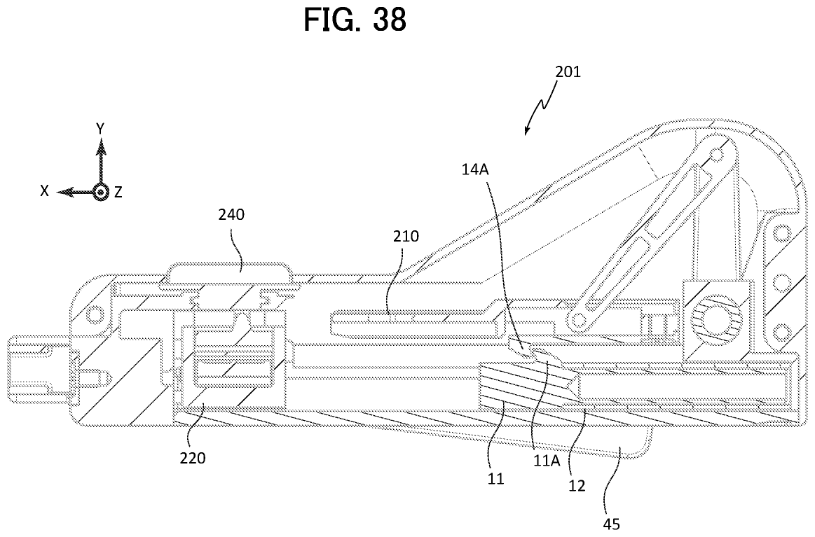

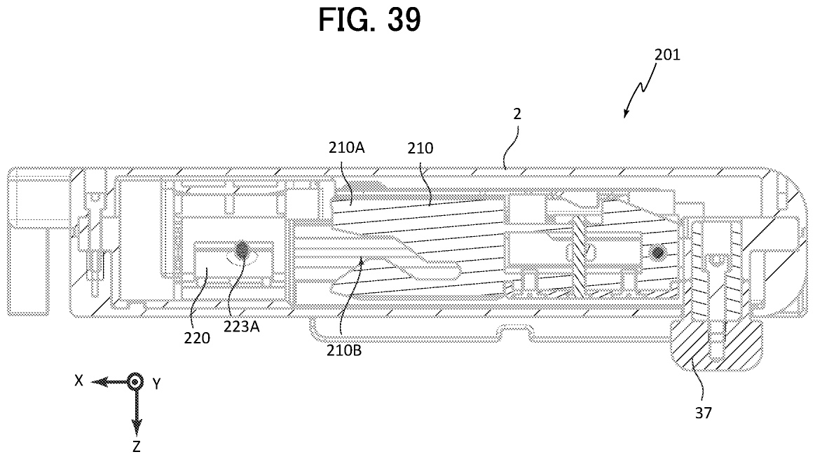

The grip open and close switch 240 includes an operation portion 240A that the technician operates and an accommodation portion 240B that accommodates the operation convex portion 223A of the grip portion 220. The grip open and close switch 240 is mounted to the housing 2 so as to be horizontally movable in the Z-axis direction. The accommodation portion 240B has a parallelepiped shape in which only a Y-axis negative direction surface is opened. The X-axis direction width of the accommodation portion 240B is a width of a moving range in the X-axis direction of the operation convex portion 223A corresponding to the moving range of the X-axis direction of the grip portion 220. Therefore, regardless of the position in the X-axis direction of the grip portion 220, it is ensured that the operation convex portion 223A is accommodated in the accommodation portion 240B. Accordingly, when the grip open and close switch 240 is operated, regardless of the position in the X-axis direction of the grip portion 220, the position of the operation convex portion 223A in the Z-axis direction can be changed according to the operation. For example, in the present embodiment, as illustrated in FIGS. 38 and 39 as described later, when the wire feeding device 201 is in the feedable state, the grip open and close switch 240 can change the position of the operation convex portion 223A. Therefore, releasing the grip of the guide wire GW by the grip portion 220 in the feedable state makes it possible to easily perform adjustment such as changing a position and direction of the guide wire GW. In the present embodiment, when the moving range of the operation convex portion 223A is regulated by the slit 210B of the slider 210, the grip open and close switch 240 cannot change the position of the operation convex portion 223A.

The following will specifically describe a use method of the wire feeding device 201 according to the third embodiment and the operation when used, with reference to the drawings. FIGS. 38 and 39 are a sectional top view and a sectional side view of the wire feeding device in the feedable state. In FIGS. 38 and 39, although the guide wire GW, the catheter 51, and the connector 60 are not illustrated, description will be made assuming that the catheter 51 and the connector are connected to the wire feeding device 201.

In the wire feeding device 201, the grip portion 220, the hammer 11, and the slider 210 are configured to interlock with each other so that gripping the guide wire GW, moving the grip portion 220 toward the distal end direction of the guide wire GW, releasing the grip of the guide wire GW, and moving the grip portion 220 toward the proximal end direction are performed in this order.

In this case, as illustrate in FIG. 31, since the convex portion 223A of the grip portion 220 is guided by the slit 210B of the extension portion 210A to the position at which the grip of the grip portion 220 is released, the wire feeding device 201 is in a state in which the grip surface 222A of the facing component 222 and the grip surface 221A of the body component 221 are separated from each other and grip of the guide wire GW by the grip portion 220 is released.

Next, when the lever 31 is turned in the R1 direction, the slider 210 slides toward the proximal end side. As illustrated in FIG. 38, since the convex portion 11A is engaged with the spring hook 14A, the hammer 11 is moved toward the proximal end side with the movement of the slider 210, so that the push spring 12 is compressed.

Here, the grip portion 220 is no longer pushed toward the X-axis positive direction by the hammer 11. Thus, the grip portion 220 slides to the rearmost end in the movable range by the energizing force of the return spring 16. As a result, the grip portion 20 moves from the frontmost end (initial position) to the rearmost end in the movable range. In the present embodiment, the return may be achieved by hooking the operation convex portion 223A on an inclined surface of the slit 210B of the slider 210 without using the return spring 16.

Further, when the lever 31 is turned in the R1 direction, the slider 210 further slides toward the proximal end side. Since a state in which the spring hook 14A is engaged with the convex portion 11A of the hammer 11 is maintained, the hammer 11 is moved toward the proximal end side with the movement of the slider 210, so that the push spring 12 is further compressed.

At this time, the operation convex portion 223A of the grip portion 220 is guided by the slit 210B of the extension portion 210A to the position at which the grip portion 220 grips, the guide wire GW between the grip surface 222A of the facing component 222 and the grip surface 221A of the body component 221 is gripped.

When the lever 31 is rotated to the position at which the slider 210 is on the most proximal end side of the moving range (feedable position), the slider 210 further slides to the proximal end side, and as similar to the case illustrated in FIGS. 12 and 13, the convex portion 45C of the injection switch 45 climbs over the convex portion 13B of the slider 210 and located in the X-axis positive direction of the convex portion 13B, so that the convex portion 45C and the convex portion 13B engage with each other. As a result, the state in which the push spring 12 is compressed is maintained, and a state in which feeding is enabled (compression maintaining state: feedable state) is established and the slider 210 is disabled to move in the X-axis positive direction.

Thereafter, when the injection switch 45 is pushed in the Y-axis positive direction by the technician, as illustrated in FIG. 14, as similar to the case illustrated in FIG. 14, the convex portion 45A pushes the convex portion 14B of the slider 210 in the Y-axis positive direction, and as similar to the case illustrated in FIG. 15, the spring hook 14A moves in the Y-axis positive direction, and a state in which the spring hook 14A is engaged with the convex portion 11A is released. The convex portion 45C moves in the positive direction further than the surface of the slider 210 in the Y-axis positive direction and does not engage with the convex portion 13B anymore, so that the slider 210 is enabled to move in the X-axis positive direction.

In this manner, the energizing force of the push spring 12 is applied at once to the movement of the hammer 11 toward the distal end direction, so that the hammer 11 moves toward the distal end direction, and the distal end side of the hammer 11 hits the proximal end side of the grip portion 220.

As a result, the grip portion 220 gripping the guide wire GW moves toward the distal end direction by the impact due to a hit with the hammer 11, and stops at the frontmost end position of the grip portion 220. Here, the grip portion 220 keeps the state of gripping the guide wire GW.

Therefore, the grip portion 220 moves from the rearmost end position to the frontmost end position while keeping the state of gripping the guide wire GW. As a result, the guide wire GW is fed to the distal end side by the distance from the rearmost end position to the frontmost end position of the grip portion 220.

Thereafter, when the technician rotates the lever 31 in a direction opposite to the R1, the slider 210 moves to the X-axis positive direction, and the wire feeding device 201 can be return to the initial state as illustrated in FIG. 30. When the guide wire GW need to be continuously fed, it is sufficient that the lever 31 is rotated in the similar manner to that described above and similar operations are performed.

As described above, in the wire feeding device 201 according to the present embodiment, it is possible to feed the guide wire GW by only an appropriate amount by applying impact force due to the energizing force accumulated in the push spring 12 on the guide wire GW. In this manner, it is possible to apply impact force on the guide wire GW, which allows the guide wire GW to penetrate an occluding object more effectively.

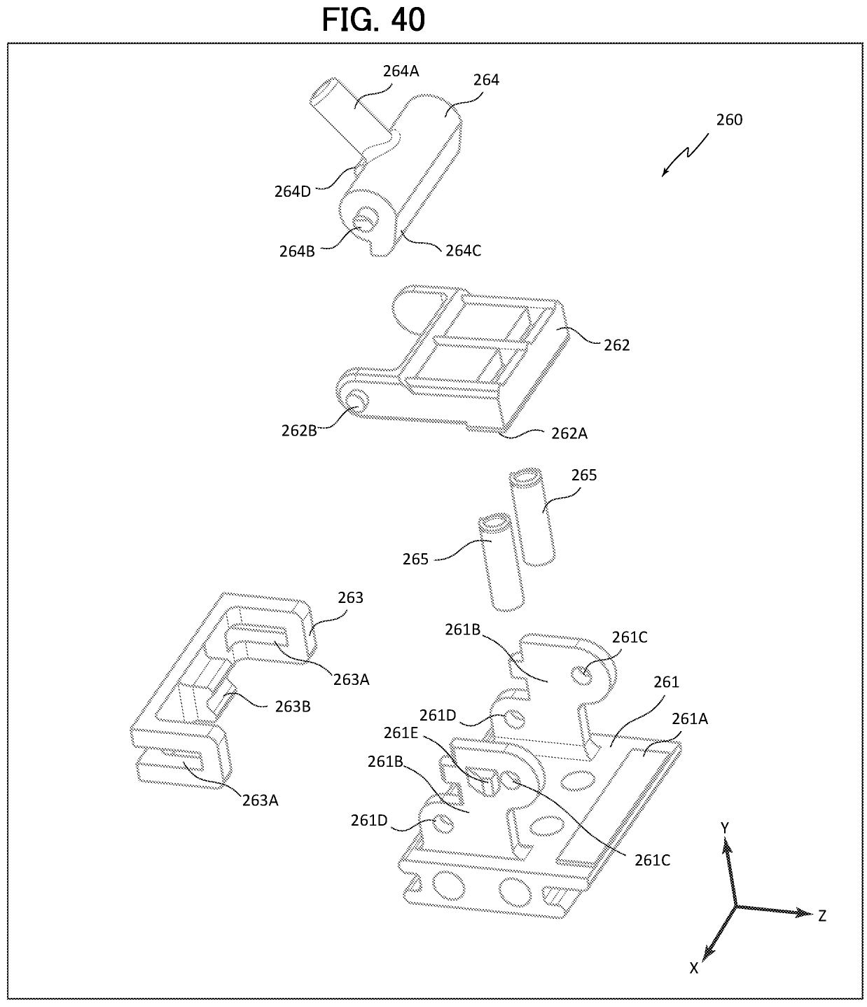

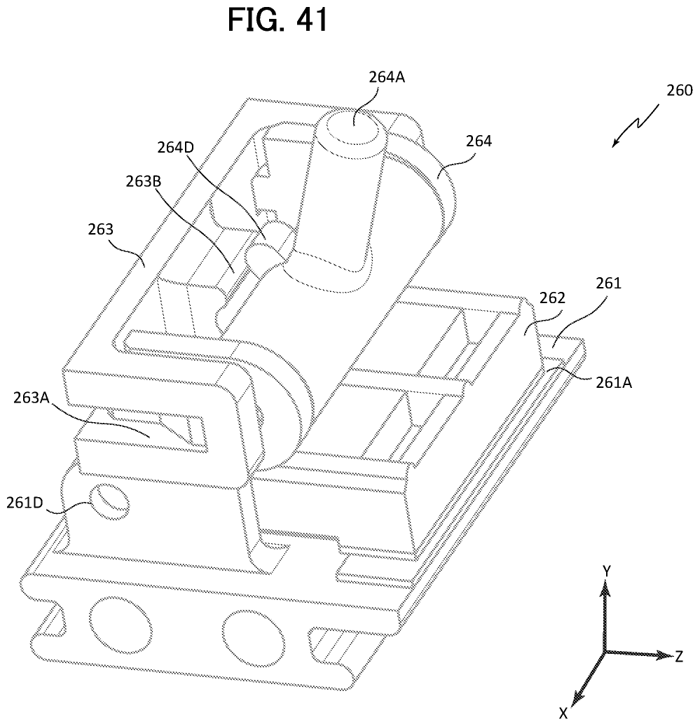

Next, a grip portion 260 according to a modification will be described with reference to the drawings. FIG. 40 is an exploded perspective view of the grip portion 260 and FIG. 41 is a perspective view of the grip portion 260 in the grip released state. The grip portion 260 can be used similarly by replacing with the grip portion 220 of the wire feeding device 201.

The grip portion 260 includes a main body portion 261, a facing component 262, a proximal surface portion 263, a cam portion 264, and a grip release spring 265.

The main body portion 261 includes a grip surface 261A and facing installation wall portion 261B. The grip surface 261A is a surface in the Y-axis negative direction that grips the guide wire GW, and for example, a rubber member may be attached to the grip surface 261A. The installation wall portion 261B includes a hole 261C to which a boss 264B on both ends of the cam portion 264 in the X-axis direction is fit, a hole 261D to which a boss 262B on both ends of the facing component 262 in the X-axis direction is fit, and a boss 261E that is fit to the proximal surface portion 263.

The cam portion 264 includes an operation convex portion 264A, a boss 264B, a contact surface 264C, and a fixation convex portion 264D. The operation convex portion 264A is a portion erected on a side surface of the cam portion 264, for operating the state of the cam portion 264. The boss 264B has a columnar shape and is formed on both ends of the cam portion 264 in the X-axis direction. The contact surface 264C is a surface that contacts a surface of the facing component 262 in the Y-axis positive direction. The cross-sectional shape of the contact surface 264C has a shape similar to that of the contact surface 223C of the cam portion 223 of the grip portion 220. The fixation convex portion 264D can engage with the fixation concave portion 263B of the proximal surface portion 263 to maintain the cam portion 264 to the state in which the grip portion 260 grips the guide wire GW.

The facing component 262 includes a grip surface 262A and a boss 262B. The grip surface 262A is a surface in the Y-axis positive direction that grips the guide wire GW, and for example, a rubber member may be attached to the grip surface 262A. The boss 262B has a columnar shape and is formed on both ends of the facing component 262 in the X-axis direction.

The proximal surface portion 263 includes a hole 263A and a fixation concave portion 263B. The hole 263A is a hole to which the boss 261E of the main body portion 261 is fitted to both ends in the X-axis direction. The fixation concave portion 263B can engage with the fixation convex portion 264D of the cam portion 264 to maintain the cam portion 264 to the state in which the grip portion 260 grips the guide wire GW.

The grip release spring 265 is arranged and energized so as to have a space between facing surfaces of the main body portion 261 and the facing component 262. In the present embodiment, when the cam portion 264 is not in the state of fixing the grip state by the grip portion 260, specifically, when the fixation convex portion 264D of the cam portion 264 does not engage with the fixation concave portion 263B of the proximal surface portion 263, the grip release spring 265 acts so as to have a space of facing surface of the main body portion 261 and the facing component 262.

Next, an assembly method of the grip portion 260 will be described.

First, in a state in which the grip cancel spring 265 is assembled to the main body portion 261, the facing component 262 and the main body portion 261 are elastically deformed to fit the boss 262B of the facing component 262 to the hole 261D of the installation wall portion 261B of the main body portion 261. Next, the cam portion 264 and the main body portion 261 is elastically deformed and the boss 264B of the cam portion 264 is fitted to the hole 261C of the installation wall portion 261B of the main body portion 261. Thereafter, the proximal surface portion 263 or the main body portion 261 are elastically deformed and the boss 261E of the main body portion 261 is fitted to the hole 263A of the proximal surface portion 263. As a result, the grip portion 260 as illustrated in FIG. 41 is completed.

### Fourth Embodiment

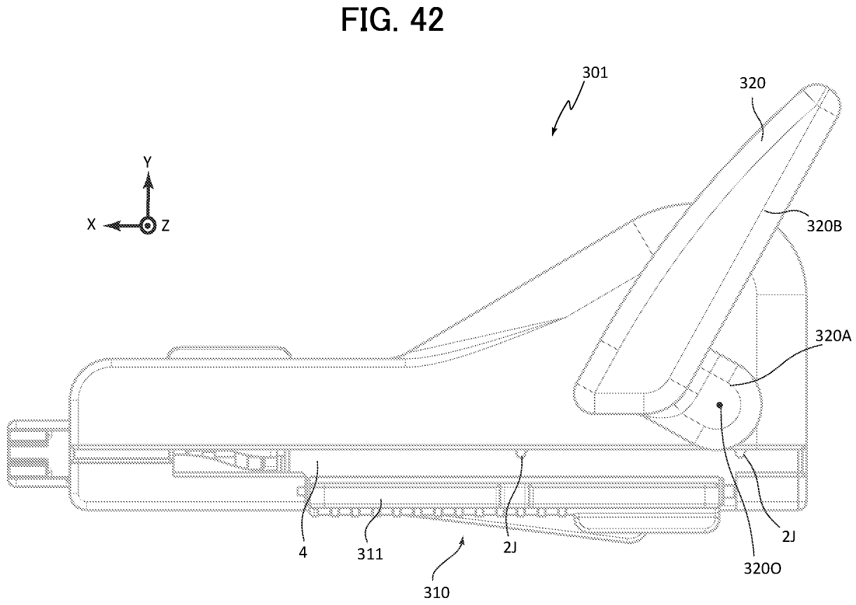

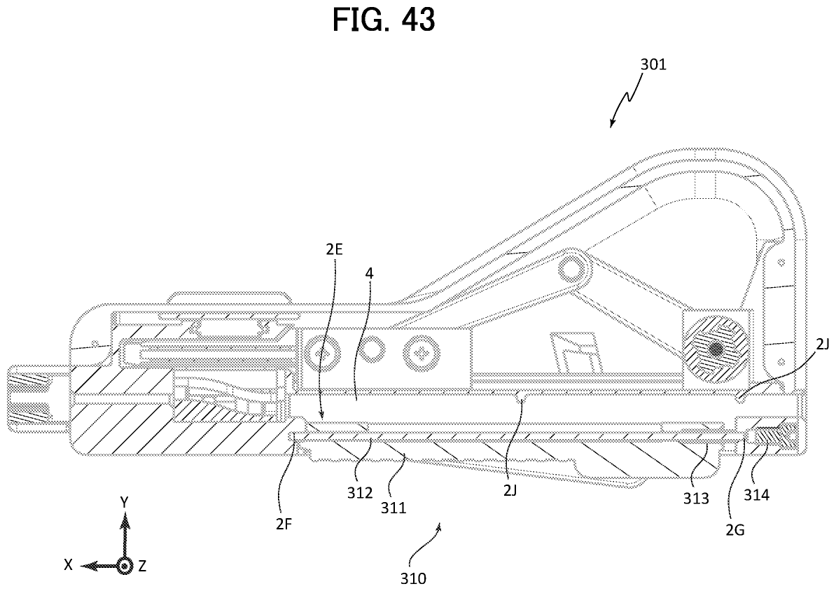

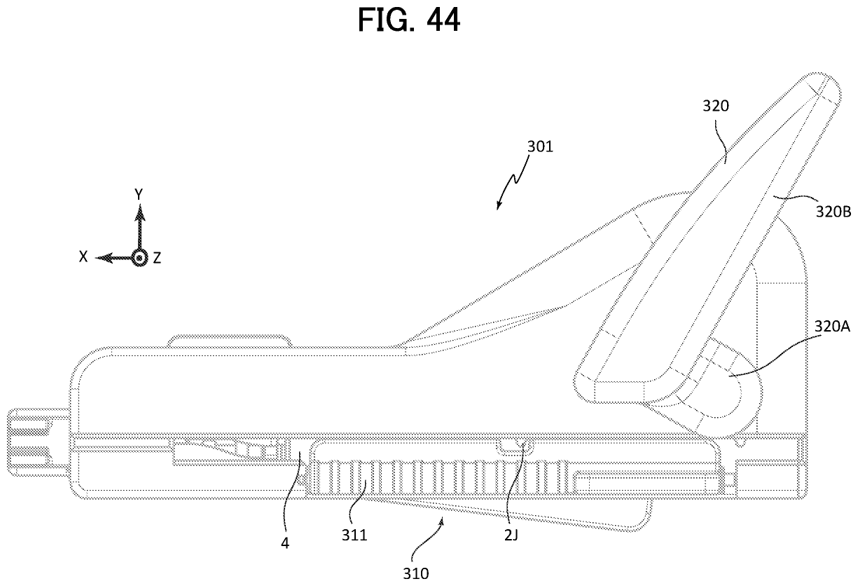

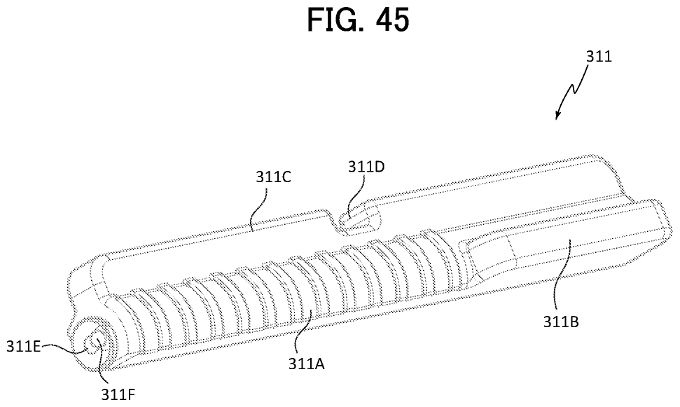

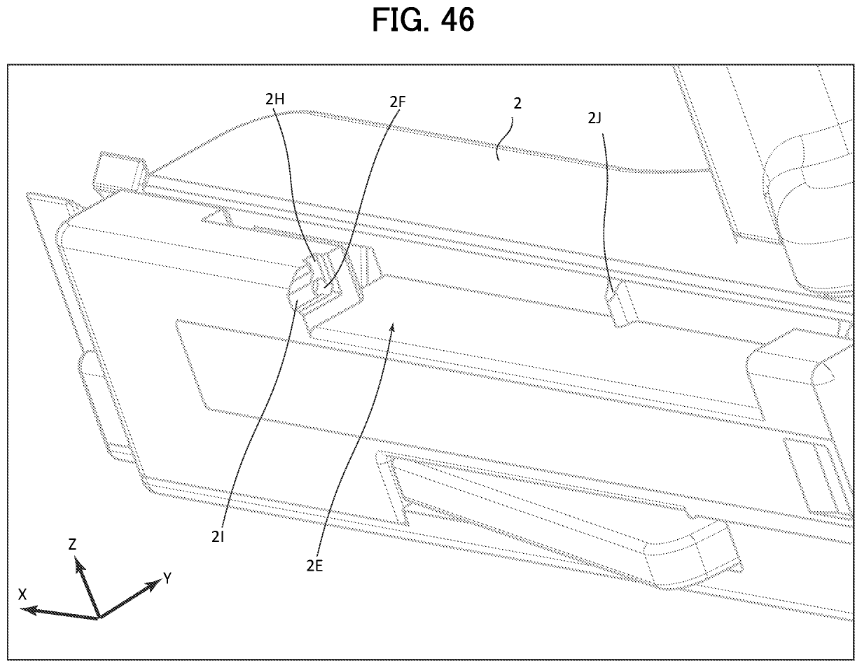

A wire feeding device according to a fourth embodiment will be described with reference to the drawings. FIGS. 42 and 43 are top view and a cross-sectional view of the wire feeding device at the time of lid opening, FIG. 44 is a top view at the time of lid closing, FIG. 45 is a perspective view of the lid, and FIG. 46 is an enlarged perspective view of an attachment part of the lid. Portions similar to those in the wire feeding device according to the first embodiment to the third embodiment are denoted by the same numbers and redundant description is omitted.

As illustrated in FIG. 42, the wire feeding device 301 further includes an open and close portion 310 for covering the guide wire accommodating portion 4, and includes a lever 320 (an example of an energizing operation portion) instead of the lever 31 used in the above-described device. As illustrated in FIG. 43, the open and close portion 310 includes a lid 311, a shaft 312, a spring 313, and a spring 314.

As illustrated in FIG. 45, the lid 311 includes a cylindrical portion 311A extending in the X-axis direction, a lid portion 311C connected to the cylindrical portion 311A for covering at least a part of the guide wire accommodating portion 4 (an example of a placement portion), and a rotation operation portion 311B for operation of rotating the lid 311.

The cylindrical portion 311A includes a through hole 311E passing through in the longitudinal direction and a boss portion 311F. The through hole 311E is a hole to which a shaft 312 for attachment is inserted. The boss portion 311F is a boss used for fixing the rotation state of the lid 311 in a predetermined state, and is arranged in a side at which the rotation operation portion 311B exists with respect to the through hole 311E. The lid portion 311C includes an opening portion 311D that prevents contact with a rib 2J.

As illustrated in FIG. 46, the housing 2 of the wire feeding device 301 is formed with an attachment area 2E, a non-through hole 2F, a through hole 2G, and a rib 2J. Furthermore, the housing 2 is formed with a groove 2H formed so as to extend in the Z-axis positive direction and a groove 2I formed so as to extend in a Y-axis negative direction, with respect to the non-through hole 2F.

As illustrated in FIG. 43, the lid 311 is attached to the housing 2 so as to be turnable around the shaft 312 by inserting the shaft 312 to the through hole 2G of the housing 2, the spring 313, the through hole 311E of the lid 311, and the non-through hole 2F. The shaft 312 is fixed to the through hole 2G of the housing 2 so as not to fall from the wire feeding device 301 by the spring 314. The spring 313 energizes the lid 311 to the distal end side (X-axis positive direction side).

When the boss portion 311F engages with the groove 2I, the open and close portion 310 is in the opened state, that is, the guide wire accommodating portion 4 is opened and the guide wire GW can be placed on the guide wire accommodating portion 4. When the boss portion 311F engages with the groove 2H, the open and close portion 310 is in the closed state, that is, at least a part of the guide wire accommodating portion 4 is closed and the guide wire GW cannot be detached from the guide wire accommodating portion 4. The lid 311 become turnable by being moved in the X-axis negative direction against the energizing of the spring 313, and the open state and the close state of the open and close portion 310 can be switched.

The lever 320 of the wire feeding device 301 includes a connection portion 320A extending from a lever rotation shaft 3200 that drives a mechanism portion, and a handle portion 320B extending from the connection portion 320A for operating by the technician with hand.

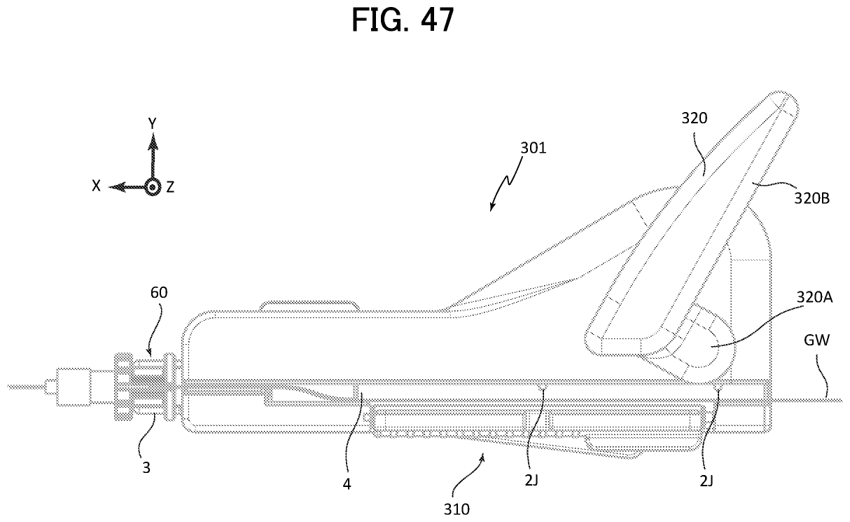

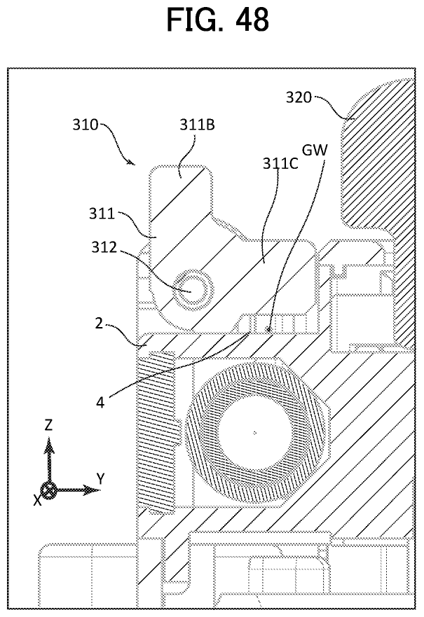

A placement method of the guide wire GW in the wire feeding device 301 will be described. FIG. 47 is a top view illustrating a state in which the guide wire GW is placed, and FIG. 48 is a sectional side view of a portion including the guide wire accommodating portion of the wire feeding device.

When the open and close portion 310 of the wire feeding device 301 is opened as illustrated in FIG. 42, as illustrated in FIG. 47, the catheter 51 is connected to the connector 60 and the connector 60 is connected to the connector connection portion 3. At this time, the guide wire GW inserted to the catheter 51 is accommodated in the guide wire accommodating portion 4. In the guide wire accommodating portion 4, the guide wire GW is placed close to the Y-axis negative direction side of the guide wire accommodating portion 4, by the rib 2J. As a result, when the open and close portion 310 is closed, the lid portion 311C of the open and close portion 310 is appropriately prevented from interfering with the guide wire GW.

Thereafter, the open and close portion 310 is closed, as illustrated in FIG. 48, the guide wire GW is accommodated in the space between the wall surface of the guide wire accommodating portion 4 and the lid portion 311C of the open and close portion 310. As a result, the guide wire GW is appropriately prevented from falling from the wire feeding device 301.

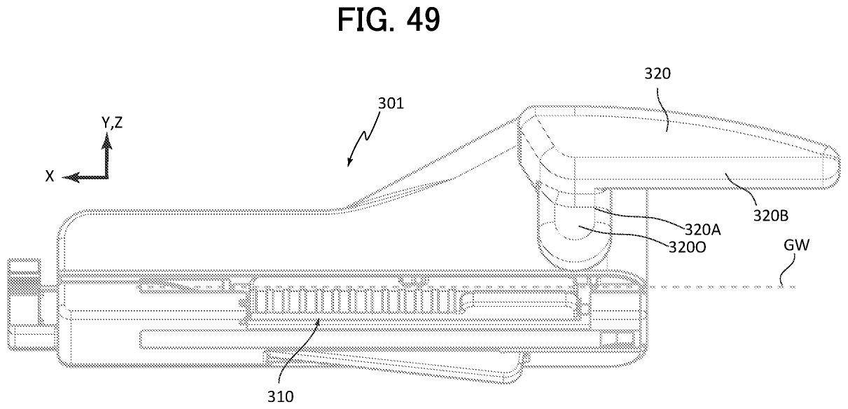

Next, the feeding operation of the guide wire GW by the lever 320 in the wire feeding device 301 will be described with reference to the drawings. FIG. 49 is a perspective view of the wire feeding device in the feedable state.

In the wire feeding device 301, in the state in which the push spring is not compressed, as illustrated in FIG. 44, the connection portion 320A of the lever 320 extends from the lever rotation axis 3200 in the X-axis positive direction and the Y-axis positive direction, and the handle portion 320B extends from the connection part with the connection portion 320A in the X-axis negative direction and the Y-axis positive direction. A bottom surface of the handle portion 320B is at a position higher than the upper surface of the housing 2 by a predetermined height or more. The predetermined height may be a height set in consideration of an assumed thickness of a finger or hand of the technician. By setting the predetermined height to be higher, the hand or finger gripping the handle portion 320B can be appropriately prevented from contacting the guide wire GW.

In the wire feeding device 301, when the lever 320 is turned in the clockwise direction from the state illustrated in FIG. 44 and the state illustrated in FIG. 49 is established, the state in which the push spring 12 is compressed, that is, the guide wire GW in the feedable state can be maintained. As described above, in the state in which the push spring 12 is compressed, the connection portion 320A extends from the lever rotation axis 3200 in the Y-axis positive direction, and the handle portion 320B extends from the connection part with the connection portion 320A in the X-axis negative direction.

As described above, in the wire feeding device 301, the moving range of the lever 320 (the connection portion 320A and the handle portion 320B) in the case where the operation for feeding the guide wire GW is performed is on the lever rotation axis 3200 side with respect to a vertical surface extending in the Z-axis direction passing the placement position of the guide wire GW. As a result, the hand or finger gripping the handle portion 320B can be appropriately prevented from contacting the guide wire GW.

### Fifth Embodiment

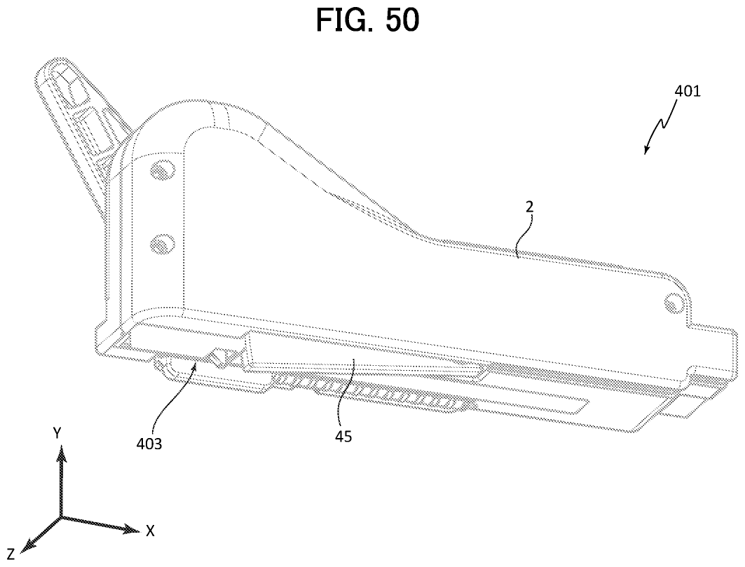

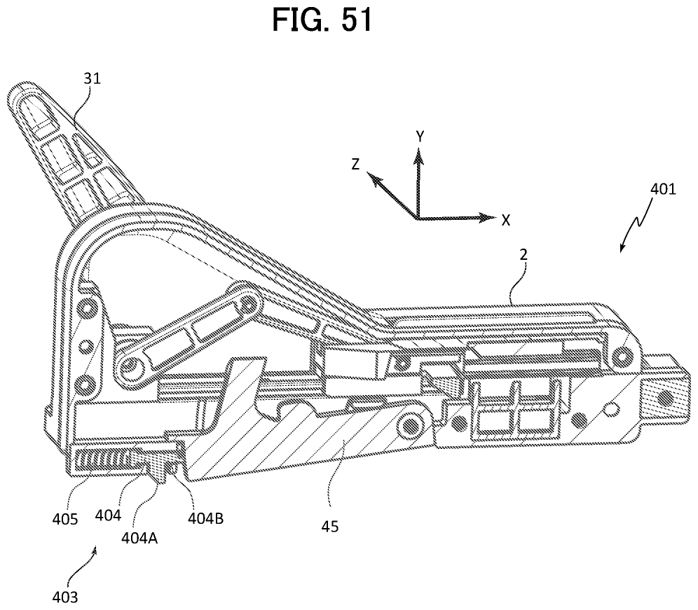

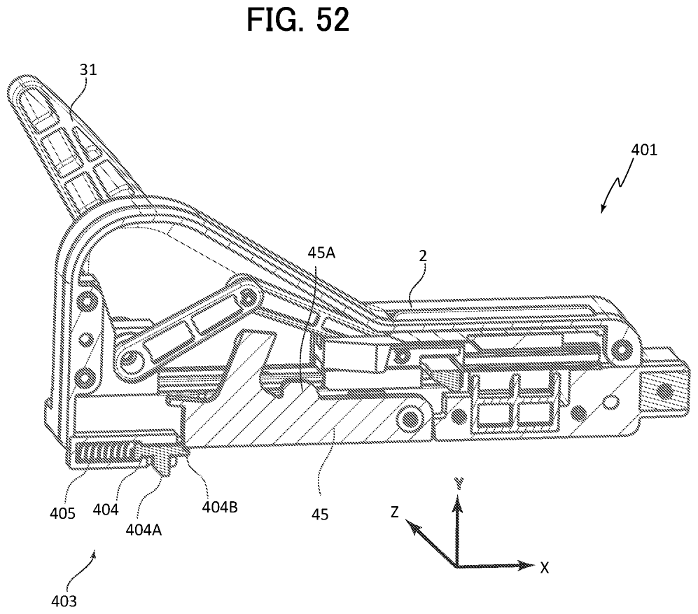

A wire feeding device 401 according to a fifth embodiment will be described with reference to the drawings. FIG. 50 is a perspective view of the wire feeding device 401, FIG. 51 is a bottom perspective view of the wire feeding device 401 at the time of non-fixation of the injection switch, and FIG. 52 is a bottom perspective view at the time of fixation. Portions similar to those in the wire feeding device according to the first embodiment to the fourth embodiment are denoted by the same numbers and redundant description is omitted.

The wire feeding device 401 further includes an operation fixing portion 403 for fixing the state in which the injection switch 45 is pushed, in the wire feeding device 301.

The operation fixing portion 403 includes a fixing switch 404, and a spring 405 that energizes the fixing switch 404 to the injection switch 45 side. The fixing switch 404 includes a convex portion 404A projecting to the outside of the housing 2 for operation by a technician, and a contact portion 404B that contacts the injection switch 45.

When the injection switch 45 is not pushed, as illustrated in FIG. 51, the contact portion 404B of the fixing switch 404 contacts the side surface of the injection switch 45 in the X-axis negative direction. In this case, the technician can push the injection switch 45.

When the injection switch 45 is pushed, as illustrated in FIG. 52, the contact portion 404B of the fixing switch 404 contacts the side surface of the injection switch 45 in the Y-axis negative side and moves in the X-axis positive direction. As a result, the injection switch 45 is disabled to turn in the Y-axis negative direction by the contact portion 404B and the pushed state is maintained.

In a case where the state in which the injection switch 45 is pushed is maintained, when the push spring 12 is compressed, the convex portion 45A of the injection switch 45 pushes the convex portion 14B, so that the spring hook 14A of the hook 14 is detached from the convex portion 11A of the hammer 11 and the hammer 11 hits the grip portion 220. Accordingly, by continuously operating the lever 31 without operating the injection switch 45 again by the technician, the guide wire GW can be continuously fed.

In a case where a state in which the injection switch 45 is pushed is maintained, when the technician performs an operation of moving the convex portion 404A of the fixing switch 404 in the X-axis negative direction, it is possible to return the state to the state in which the injection switch 45 is not pushed as illustrated in FIG. 51.

The technology disclosed in this specification is not limited to the above-described embodiments, and can be modified in various forms without departing from the gist thereof. For example, the following modifications are also possible.

In the above-described embodiments, metal springs are used as the push springs 12. However, other kinds of elastic bodies such as rubber cords and plate springs may be used. The material of the elastic body may be a resin material. In the above-described embodiment, the hammers 11 are also made of metal. However, they may be made of a resin material, similarly to the push springs.

The hammers 11 are moved to compress the push springs 12, thereby increasing the energizing force on the hammers 11 toward the distal end direction. However, an elastic body may be provided to extend with the movement of the hammers 11 toward the proximal end side, so that the energizing force is increased by the extension of the elastic body, for example. Alternatively, for example, the push spring 12 may directly move the grip portions 20, 220, 260 to the distal end direction by using the push spring 12 without using the hammer 11.

It is possible to provide a mechanism for adjusting the movable range of the grip portions 20, 220, 260 in the X-axis direction, such as a mechanism for moving the position in the X-axis direction of the wall that determines the movable range, for example. In this manner, it is possible to easily and appropriately adjust the wire feeding amount by the wire feeding device.

It is possible to provide a mechanism for adjusting the compression amount of the push springs 12 in the initial state, such as a mechanism for moving the position of the wall on the proximal end side of the push springs 12, for example. In this manner, it is possible to easily and appropriately adjust the impact force applied on the grip portions 20, 220, 260 by the hammers 11 of the wire feeding device. In the above-mentioned embodiment, it is assumed that the push spring 12 is not compressed at all in the initial state. However, the push spring 12 may be slightly compressed in the initial state.

The technician manually turns the lever 31, 320 to feed the guide wire GW. However, a power-operated motor may be used to feed the guide wire GW. For example, the link 35 may be turned by the power of a motor. In this case, the motor may be stopped when the link 35 is turned by a predetermined angle. For example, a switch for driving the motor may be provided, so that when this switch is pushed once, the motor may be driven to turn the link 35 by a predetermined angle.

The configurations of the wire feeding devices according to the above-described embodiments may be combined. For example, the grip portion 20 in the second embodiment may be replaced with the grip portions 220, 260 in the third embodiment. The lever 31 in the first embodiment and the second embodiment may be replaced with the lever 320 in the fourth embodiment. The open and close portion 310 in the fourth embodiment may be provided in the wire feeding device according to the first embodiment and the second embodiment. The operation fixing portion 403 in the fifth embodiment may be provided in the wire feeding device according to the first embodiment to the third embodiment.

The disclosed embodiments are not limited to the configurations of the above-described embodiments, but are defined by the terms of the claims and are intended to include any modifications within the scope and meaning equivalent to the terms of the claims.

1. A wire feeding device for feeding a wire in a distal end direction, the wire feeding device comprising:
a grip portion configured to grip and release the wire and to move in the distal end direction and a proximal end direction;
an elastic body configured to bias the grip portion toward the distal end direction; and
an energizing portion configured to deform the elastic body and increase a biasing force of the elastic body toward the distal end direction, wherein
when deformation of the elastic body by the energizing portion is released, the wire gripped by the grip portion is fed in the distal end direction by the biasing force of the elastic body, and
the wire feeding device is configured to be operated in (i) a first mode in which the wire is continuously fed in the distal end direction according to a first predetermined operation and (ii) a second mode in which the wire is fed once in the distal end direction according to a second predetermined operation.

2. The wire feeding device according to claim 1, wherein
in the first mode, a series of operations is repeatedly performed according to the first predetermined operation in order of (a), (b), and (c):
(a) gripping of the wire by the grip portion and deformation of the elastic body by the energizing portion,
(b) releasing of the deformation of the elastic body and feeding of the grip portion gripping the wire to the distal end side by the biasing force of the elastic body, and
(c) releasing of the wire by the grip portion and movement of the grip portion to a proximal end side, and
in the second mode, (a) is performed and then (b) is performed according to the second predetermined operation.

3. The wire feeding device according to claim 1, further comprising
an operation portion configured to switch between the first mode and the second mode.

4. The wire feeding device according to claim 3, further comprising
a deformation maintaining portion configured to maintain a deformation state of the elastic body that is in a predetermined state due to an increase in the biasing force by the energizing portion; and
a maintaining state change portion configured to change to a state in which maintaining of the deformation state of the elastic body by the deformation maintaining portion is disabled, wherein
the operation portion is configured to set the maintaining state change portion to the state in which maintaining of the deformation state of the elastic body by the deformation maintaining portion is disabled, and
in a case where the deformation state of the elastic body that is in the predetermined state due to an increase in the biasing force by the energizing portion is maintained by the deformation maintaining portion, when the operation portion is operated to set the maintaining state change portion to the state in which maintaining of the deformation state of the elastic body by the deformation maintaining portion is disabled, the grip portion is moved in the distal end direction by the biasing force of the elastic body that is disabled to maintain the deformation state and the wire gripped by the grip portion is fed in the distal end direction.

5. The wire feeding device according to claim 4, wherein
the wire feeding device is configured to be operated in the first mode when the operation portion sets the maintaining state change portion to the state in which maintaining of the deformation state of the elastic body by the deformation maintaining portion is disabled and maintains the state.

6. The wire feeding device according to claim 5, further comprising
an operation fixing portion configured to fix the operation portion to the state in which the maintaining state change portion is set to the state in which maintaining of the deformation state of the elastic body by the deformation maintaining portion is disabled.

7. The wire feeding device according to claim 1, further comprising
a grip operation portion configured to operate a state of gripping of the wire by the grip portion.

8. A wire feeding device for feeding a wire in a distal end direction, the wire feeding device comprising:
a grip portion configured to grip and release the wire and to move in the distal end direction and a proximal end direction;
an elastic body configured to bias the grip portion toward the distal end direction;
an energizing portion configured to deform the elastic body and increase a biasing force of the elastic body toward the distal end direction; and
a release portion that releases a deformation state of the elastic body with the biasing force increased by the energizing portion, wherein
the wire gripped by the grip portion is fed toward the distal end direction by moving the grip portion in the distal end direction by the biasing force of the elastic body whose deformation state is released by the release portion, and
the wire feeding device further comprises a grip operation portion configured to operate a state of gripping of the wire by the grip portion.

9. The wire feeding device according to claim 8, wherein
the release portion includes
a deformation maintaining portion configured to maintain the deformation state of the elastic body that is in a predetermined state due to an increase in the biasing force by the energizing portion, and
a maintaining state change portion configured to change a state to a state in which maintaining of the deformation state of the elastic body by the deformation maintaining portion is disabled, and
the grip operation portion is configured to operate the grip portion gripping the wire to a state of grip release when the deformation state of the elastic body is maintained by the deformation maintaining portion.

10. The wire feeding device according to claim 1, further comprising
a hitting portion that is arranged on a proximal end side of the grip portion, is configured to move in an axial direction of the wire, and is configured to come into contact with and separate from the grip portion.

11. The wire feeding device according to claim 1, wherein
the grip portion includes a first clamping portion and a second clamping portion that clamp the wire, and a cam portion configured to adjust a distance between the first clamping portion and the second clamping portion.

12. The wire feeding device according to claim 1, further comprising
a placement portion on which the wire to be gripped by the grip portion is placed, the placement portion having an entire surface in a predetermined direction being opened over a longitudinal direction of the wire, and an open and close portion configured to open and close at least a part of the opened surface in the predetermined direction of the placement portion.

13. The wire feeding device according to claim 1, further comprising
an energizing operation portion configured to be manually operated to increase the biasing force of the elastic body by the energizing portion, wherein
the energizing operation portion includes a handle portion configured to be manually operated to a position offset from a placement position of the wire.
